(19) United States
(12) Patent Application Publication
(10) Pub. No.: US 2015/0044772 A1
(43) Pub. Date: Feb. 12, 2015

(54) CRISPR/CAS SYSTEM-BASED NOVEL FUSION PROTEIN AND ITS APPLICATIONS IN GENOME EDITING

(71) Applicant: Sage Labs, Inc., St. Louis, MO (US)

(72) Inventor: Guojun Zhao, St. Louis, MO (US)

(21) Appl. No.: 14/455,603

(22) Filed: Aug. 8, 2014

Related U.S. Application Data
(60) Provisional application No. 61/864,111, filed on Aug. 9, 2013.

(51) Int. Cl. C12N 15/01; C12N 9/22

(52) U.S. Cl. CPC C12N 15/01; C12N 9/22; C07K 2319/00
USPC 435/462; 435/188; 536/23.2; 435/320.1; 435/252.3; 435/468; 435/471; 435/325; 435/419; 435/366; 435/348; 435/354; 435/353; 435/352; 435/351; 435/350; 435/349

(57) ABSTRACT

An inactive CRISPR/Cas system-based fusion protein and its applications in gene editing are disclosed. More particularly, chimeric fusion proteins including an inCas fused to a DNA modifying enzyme and methods of using the chimeric fusion proteins in gene editing are disclosed. The methods can be used to induce double-strand breaks and single-strand nicks in target DNAs, to generate gene disruptions, deletions, point mutations, gene replacements, insertions, inversions and other modifications of a genomic DNA within cells and organisms.

### CROSS REFERENCE TO RELATED APPLICATION(S)

This application claims benefit of U.S. Provisional Patent Application Ser. No. 61/864,111, filed Aug. 9, 2013, the entire disclosure of which is herein incorporated by reference.

### INCORPORATION OF SEQUENCE LISTING

A paper copy of the Sequence Listing and a computer readable form of the sequence containing the file named 3362304_ST25.txt, which is 130,453 bytes in size (as measured in MS-DOS), are provided herein and are herein incorporated by reference. This Sequence Listing consists of SEQ ID NOS: 1-40.

### BACKGROUND

1. Field of the Invention

The present disclosure is directed to chimeric fusion proteins and methods of gene editing using the chimeric fusion proteins. The chimeric fusion proteins of the present disclosure include a catalytically inactive CRISPR associated protein (“inCas” or “dCas”) domain fused to a DNA modifying domain. The methods include introducing a chimeric fusion protein into a cell or an organism where the chimeric fusion protein induces a DNA modification in a target DNA.

2. Description of the Related Art

Engineered sequence-specific nucleases provide powerful tools for genome editing. These nucleases enable investigators to manipulate virtually any gene in a diverse range of cell types and organisms. Currently, the most widely used engineered nucleases are Zinc Finger Nucleases (ZFNs) and Transcription Activator-Like Effector Nucleases (TALENs). These engineered fusion nucleases consist of a sequence-specific DNA binding domain and the FokI nuclease domain. FokI is a bacterial type IIS restriction endonuclease that is naturally found in Flavobacterium okeanokoites. An important feature of the FokI nuclease domain is that it cleaves DNA only as a dimer. Upon binding to specific DNA sequences flanking a desired cleavage site, two distinct, paired ZFN or TALEN fusion protein monomers form the FokI dimer and thus induce double-strand breaks (DSBs) that stimulate error-prone nonhomologous end joining (NHEJ) or homologous recombination (HR) at specific genomic locations. While these engineered fusion nucleases have been successfully used to mediate precise genetic modifications in diverse types of cells and organisms, construction of specific, high-affinity ZFNs and TALENs remains difficult. For example, different fusion nucleases must be constructed to target different sites. In many cases it can also require using time-consuming and labor-intensive systems that are not readily adopted by non-specialty laboratories.

Recently, the prokaryotic type II CRISPR (clustered regularly interspaced short palindromic repeats)/Cas (CRISPR associated) adaptive immune system has emerged as an alternative to ZFNs and TALENs for inducing targeted genetic alterations (Jinek et al. Science 2012 337:816-21). In bacteria, the CRISPR system provides acquired immunity against invading foreign DNA via RNA-guided DNA cleavage. Short fragments of foreign DNA sequences, termed protospacers, integrate into the CRISPR locus of the bacterial genome. The transcribed CRISPR RNAs (crRNAs) anneal to trans-activating crRNAs (tracrRNA) and these crRNAs-tracrRNAs hybrids direct sequence-specific cleavage and silencing of pathogenic DNA by Cas proteins.

One well-studied CRISPR/Cas systems is the CRISPR/Cas9 system from Streptococcus pyogenes. The Cas9 is a crRNA guided double-strand DNA endonuclease with RuvC and HNH active site motifs each of which cleaves one strand within the target DNA. Point mutations of these two active sites abolish CRISPR/Cas9 endonuclease activity, but still retain Cas9 DNA binding specificity. This specificity of the Cas9 endonuclease is mediated by an engineered single guide RNA (sgRNA) that mimics the natural crRNA-tracrRNA hybrid. Target DNA recognition and cleavage uses a sequence match between the target site and the 12-20 nucleotides (nt) of the sgRNA sequence (the crRNA part), as well as a protospacer adjacent motif (PAM) located near the target site. Therefore, reprogramming of Cas9 DNA specificity does not require changes in the Cas9 protein but only in the sequence of the sgRNAs, which makes the CRISPR/Cas9 system a very simple tool for genome editing. Indeed, this RNA guided DNA cleavage system has been used to edit genomes in different model systems including different types of cells and model organisms such as yeast, zebrafish, Drosophila, C. elegans, mouse, rat, and livestock.

Nevertheless, while this CRISPR/Cas9 system is efficient and easy to handle, its specificity only depends on the 12-20 nt sequence in the single guide RNA (sgRNA) and a PAM sequence. Furthermore, a few mutations in this 12-20 nt sequence region do not significantly affect Cas9 cleavage. Very recently, significant off-target effects have been revealed in human cells. These off-target sites identified in human cells contain up to five base pair mismatches and many were mutagenized with frequencies comparable to, or even higher than, those at the desired target site.

Accordingly, there is a need for CRISPR/Cas-based novel systems with high specificity, especially for use in cells and organisms.

### SUMMARY

In one aspect, the present disclosure is directed to a chimeric fusion protein including a DNA modifying domain fused to a catalytically inactive CRISPR associated (“inCas”, or “dCas”) domain. To be consistent with current literature, the “dCas9” is used for catalytically inactive Cas9 protein in the rest of this disclosure.

In another aspect, the present disclosure is directed to an isolated nucleic acid comprising a nucleotide sequence encoding a chimeric fusion protein including a DNA modifying domain fused to a catalytically inactive CRISPR associated (dCas) domain.

In another aspect, the present disclosure is directed to a vector comprising a nucleotide sequence encoding a chimeric fusion protein including a DNA modifying domain fused to a catalytically inactive CRISPR associated (dCas) domain.

In another aspect, the present disclosure is directed to a cell comprising a vector that comprises a nucleic acid sequence encoding a chimeric fusion protein including a DNA modifying domain fused to a catalytically inactive CRISPR associated (dCas) domain.

In another aspect, the present disclosure is directed to a cell comprising a nucleic acid sequence encoding a chimeric fusion protein a DNA modifying domain fused to a catalytically inactive CRISPR associated (dCas) domain.

In another aspect, the present disclosure is directed to an organism including a vector that comprises a nucleic acid sequence encoding a chimeric fusion protein including a DNA modifying domain fused to a catalytically inactive CRISPR associated (dCas) domain.

In another aspect, the present disclosure is directed to an organism comprising a nucleic acid sequence encoding a chimeric fusion protein including a DNA modifying domain fused to a catalytically inactive CRISPR associated (dCas) domain.

In another aspect, the present disclosure is directed to a chimeric fusion protein comprising a FokI domain fused to a catalytically inactive Cas9 (dCas9) domain.

In another aspect, the present disclosure is directed to an isolated nucleic acid comprising a nucleotide sequence encoding a chimeric fusion protein including a FokI domain fused to a dCas9 domain.

In another aspect, the present disclosure is directed to a vector comprising a nucleotide sequence encoding a chimeric fusion protein including a FokI domain fused to a dCas9 domain.

In another aspect, the present disclosure is directed to a cell comprising a vector that comprises a nucleotide sequence encoding a FokI domain fused to a dCas9 domain.

In another aspect, the present disclosure is directed to a cell comprising a nucleic acid sequence encoding a chimeric fusion protein including a FokI domain fused to a dCas9 domain.

In another aspect, the present disclosure is directed to an organism comprising a vector that comprises a nucleotide sequence encoding a chimeric fusion protein including a FokI domain fused to a dCas9 domain.

In another aspect, the present disclosure is directed to an organism comprising a nucleic acid sequence encoding a chimeric fusion protein including a FokI domain fused to a dCas9 domain.

In another aspect, the present disclosure is directed to a method of genome editing. The method includes introducing at least two chimeric fusion protein monomers into a cell, wherein the at least two chimeric fusion protein monomers each includes a DNA modifying domain fused to a dCas domain; introducing a first guide RNA (sgRNA) and a second guide RNA (sgRNA) into the cell, wherein the first sgRNA and the second sgRNA comprise an at least 12-20 nucleotide sequence complementary to two adjacent target DNA nucleotide sequences and wherein the first sgRNA forms a first complex with one chimeric fusion protein monomer and wherein the second sgRNA forms a second complex with one chimeric fusion protein monomer to direct the at least two chimeric fusion protein monomers to the adjacent target DNA nucleotide sequences, wherein the DNA modifying domains of the two chimeric fusion protein monomers form a functional DNA modifying domain dimer and induce a DNA modification in the target DNA.

In another aspect, the present disclosure is directed to a method of genome editing. The method includes introducing at least two chimeric fusion protein monomers into an organism, wherein the at least two chimeric fusion protein monomers each includes a DNA modifying domain fused to a dCas domain; introducing a first guide RNA (sgRNA) and a second guide RNA (sgRNA) into the organism, wherein the first sgRNA and the second sgRNA comprise an at least 12 to 20 nucleotide sequence complementary to two adjacent target DNA nucleotide sequences and wherein the first sgRNA forms a first complex with one chimeric fusion protein monomer and wherein the second sgRNA forms a second complex with one chimeric fusion protein monomer to direct the at least two chimeric fusion protein monomers to the adjacent target DNA nucleotide sequences, wherein the DNA modifying domains of the two chimeric fusion protein monomers form a functional DNA modifying domain dimer and induce a DNA modification in the target DNA.

In another aspect, the present disclosure is directed to a method of genome editing. The method includes introducing at least two chimeric fusion protein monomers into a cell, wherein the at least two chimeric fusion protein monomers each comprises a FokI domain fused to a dCas9 domain; introducing a first guide RNA (sgRNA) and a second guide RNA (sgRNA) into the cell, wherein the first sgRNA and the second sgRNA comprise an at least 12-20 nucleotide sequence complementary to two adjacent target DNA nucleotide sequences and wherein the first sgRNA forms a first complex with one chimeric fusion protein monomer and wherein the second sgRNA forms a second complex with one chimeric fusion protein monomer to direct the at least two chimeric fusion protein monomers to the adjacent target DNA nucleotide sequences, wherein the FokI domains of the two chimeric fusion protein monomers form a FokI dimer and induce at least one break in the target DNA.

In another aspect, the present disclosure is directed to a method of inducing a double-strand break in a target DNA in a cell. The method includes introducing at least two chimeric fusion protein monomers into a cell, wherein the at least two chimeric fusion protein monomers each comprises a FokI domain fused to a dCas9 domain; introducing a first guide RNA (sgRNA) and a second guide RNA (sgRNA) into the cell, wherein the first sgRNA and the second sgRNA comprise an at least 12-20 nucleotide sequence complementary to two adjacent target DNA nucleotide sequences and wherein the first sgRNA forms a first complex with one chimeric fusion protein monomer and wherein the second sgRNA forms a second complex with one chimeric fusion protein monomer to direct the at least two chimeric fusion protein monomers to the adjacent target DNA nucleotide sequences, wherein the FokI domains of the two chimeric fusion protein monomers form a FokI dimer and induce double-strand breaks in the target DNA.

In another aspect, the present disclosure is directed to a method of inducing a double-strand break in a target DNA in an organism. The method includes introducing at least two chimeric fusion protein monomers into an organism, wherein the at least two chimeric fusion protein monomers each comprises a FokI domain fused to a dCas9 domain; introducing a first guide RNA (sgRNA) and a second guide RNA (sgRNA) into the organism, wherein the first sgRNA and the second sgRNA comprise an at least 12-20 nucleotide sequence complementary to two adjacent target DNA nucleotide sequences and wherein the first sgRNA forms a first complex with one chimeric fusion protein monomer and wherein the second sgRNA forms a second complex with one chimeric fusion protein monomer to direct the at least two chimeric fusion protein monomers to the adjacent target DNA nucleotide sequences, wherein the FokI domains of the two chimeric fusion protein monomers form a FokI dimer and induce double-strand breaks in the target DNA.

In another aspect, the present disclosure is directed to a method of inducing a double-strand break in a target DNA in a cell. The method includes introducing a chimeric fusion protein monomer that comprises a FokI domain fused to a dCas9 domain into a cell; introducing at least one guide RNA (sgRNA) into the cell, wherein the sgRNA comprises an at least 12-20 nucleotide sequence complementary to a sequence in a target DNA, and wherein the sgRNA forms a complex with the chimeric fusion protein monomer; wherein the sgRNA guides binding of the chimeric fusion protein monomer to the target DNA; and introducing a nuclease into the cell, wherein the nuclease comprises a FokI domain and binds to the adjacent DNA sequence of the sgRNA target site; wherein the FokI domain of the chimeric fusion protein monomer and the FokI domain of the nuclease form a FokI dimer and induces double-strand breaks in the target DNA.

In another aspect, the present disclosure is directed to a method of inducing a double-strand break in a target DNA in a cell. The method includes introducing a chimeric fusion protein monomer that comprises a FokI domain fused to a dCas9 domain (FokI-dCas9) into a cell; introducing at least one guide RNA (sgRNA) into the cell, wherein the sgRNA comprises an at least 12-20 nucleotide sequence complementary to a sequence in a target DNA and wherein the sgRNA forms a complex with the FokI-dCas9 chimeric fusion protein monomer; wherein the sgRNA guides binding of the FokI-dCas9 chimeric fusion protein monomer to the target DNA; and introducing a nuclease into the cell, wherein the nuclease comprises a FokI domain and binds to the adjacent DNA sequence of the sgRNA target site; wherein the nuclease is a zinc finger nuclease (ZFN), wherein the FokI domain of the FokI-dCas9 chimeric fusion protein monomer and the FokI domain of the ZFN form a FokI dimer and induces a double-strand break in the target DNA.

In another aspect, the present disclosure is directed to a method of inducing a double-strand break in a target DNA in a cell. The method includes introducing a chimeric fusion protein monomer that comprises a FokI domain fused to a dCas9 domain (FokI-dCas9) into a cell; introducing a guide RNA (sgRNA) into the cell, wherein the sgRNA comprises an at least 12-20 nucleotide sequence complementary to a sequence in a target DNA and wherein the sgRNA forms a complex with the FokI-dCas9 chimeric fusion protein monomer; wherein the sgRNA guides binding of the FokI-dCas9 chimeric fusion protein monomer to the target DNA; and introducing a nuclease into the cell, wherein the nuclease comprises a FokI domain; wherein the nuclease is a transcription activator-like effector nuclease (TALEN), wherein the FokI domain of the FokI-dCas9 chimeric fusion protein monomer and the FokI domain of the TALEN form a FokI dimer and induces double-strand breaks in the target DNA.

In another aspect, the present disclosure is directed to a method of inducing a double-strand break in a target DNA in an organism. The method includes introducing at least one chimeric fusion protein monomer that comprises a FokI domain fused to a dCas9 domain (FokI-dCas9) into an organism; introducing at least one guide RNA (sgRNA) into the organism, wherein the sgRNA comprises an at least 12-20 nucleotide sequence complementary to a sequence in a target DNA and wherein the sgRNA forms a complex with the chimeric fusion protein monomer; wherein the sgRNA guides binding of a FokI-dCas9 chimeric fusion protein monomer to the target DNA; and introducing a nuclease into the organism, wherein the nuclease comprises a FokI domain and binds to the adjacent DNA sequence of the sgRNA target site; wherein the FokI domain of the FokI-dCas9 chimeric fusion protein monomer and the FokI domain of the nuclease form a FokI dimer and induces double-strand breaks in the target DNA.

In another aspect, the present disclosure is directed to a method of inducing a double-strand break in a target DNA in an organism. The method includes introducing a chimeric fusion protein monomer that comprises a FokI domain fused to dCas9 domain (FokI-dCas9) into an organism; introducing at least one guide RNA (sgRNA) into the organism, wherein the sgRNA comprises an at least 12-20 nucleotide sequence complementary to a sequence in a target DNA and wherein the sgRNA forms a complex with the FokI-dCas9 chimeric fusion protein monomer; wherein the sgRNA guides binding of the FokI-dCas9 chimeric fusion protein monomer to the target DNA; and introducing a different nuclease into the organism, wherein the different nuclease comprises a FokI domain and binds to the adjacent DNA sequence of the sgRNA target site; wherein the nuclease is a zinc finger nuclease (ZFN), wherein the FokI domain of the FokI-dCas9 chimeric fusion protein monomer and the FokI domain of the ZFN form a FokI dimer and induces double-strand breaks in the target DNA.

In another aspect, the present disclosure is directed to a method of inducing a double-strand break in a target DNA in an organism. The method includes introducing at least one chimeric fusion protein monomer that comprises a FokI domain fused to a dCas9 domain (FokI-dCas9) into an organism; introducing at least one guide RNA (sgRNA) into the organism, wherein the sgRNA comprises an at least 12-20 nucleotide sequence complementary to a sequence in a target DNA and wherein the sgRNA forms a complex with the FokI-dCas9 chimeric fusion protein monomer; wherein the sgRNA guides binding of the FokI-dCas9 chimeric fusion protein monomer to the target DNA; and introducing a different nuclease into the organism, wherein the different nuclease comprises a FokI domain and binds to the adjacent DNA sequence of the sgRNA target site; wherein the nuclease is a TALEN, wherein the FokI domain of the FokI-dCas9 chimeric fusion protein monomer and the FokI domain of the TALEN form a FokI dimer and induces double-strand breaks in the target DNA.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a schematic illustration showing two FokI-Linker-dCas9 (FokI-dCas9) fusion proteins binding to a target DNA and inducing a double strand break. A pair of sgRNAs (sgRNA1 and sgRNA2) targeting two adjacent sites on the target DNA direct two monomeric FokI-dCas9 fusion proteins to the target DNA. When the two monomeric FokI-dCas9 fusion proteins are in close proximity, a FokI dimer forms, and induces a DSB in the target DNA. The bigger oval represents the dCas9 domain of the FokI-dCas9 fusion protein; the smaller oval represents the FokI endonuclease domain of the FokI-dCas9 fusion protein; and the thick solid line represents the linker between FokI and dCas9 domains. The two longer parallel lines represent a double stranded target DNA. A first sgRNA (sgRNA1) includes about a 16-20 nucleotide sequence complementary to one site on the upstream side of a target DNA, while a second sgRNA (sgRNA2) includes about a 16-20 nucleotide sequence complementary to another site on the downstream side of the target DNA. The two target sites of the sgRNAs are in adjacent regions, and are on the complementary strands of the target DNA (as shown). The two PAMs are outside of the two sgRNA target sites. The resulting target DNA with the double-strand breaks (DSBs) induced by the FokI-dCas9 dimer (in the presence of two sgRNAs) can be repaired via either error-prone nonhomologous end joining (NHEJ) or homologous recombination (HR) to mediate genetic modifications.

FIG. 2 is a schematic illustration showing a FokI-dCas9 and ZFN heterodimer-mediated genome editing. A Zinc Finger Nuclease (ZFN) and a single sgRNA guided FokI-dCas9 fusion protein are targeted to two adjacent sites on a genomic DNA, and form a FokI-based dimer and create a DNA double strand break that is repaired by either NHEJ or HR pathways. The FokI DNA cleavage domain in the dimer can be the same or different ones that can form a functional dimer.

FIG. 3 is a schematic illustration showing a FokI-dCas9 and TALEN heterodimer-mediated genome editing. A TALEN and a single sgRNA guided Fok-dCas9 fusion protein are targeted to two adjacent sites on a genomic DNA, and form a FokI-based dimer and create a DNA double strand break that is repaired by either NHEJ or HR pathways. The FokI DNA cleavage domain in the dimer can be the same or different ones that can form a functional dimer.

FIG. 4 is schematic representation of Cas9, dCas9, FokI-dCas9, and dCas9-FokI fusion proteins and their variants. A FokI-dCas9 fusion protein comprises a FokI DNA cleavage domain, a catalytically inactive Cas9 domain or a fragment of a dCas9, at least one nuclear localization signal (NLS) and a Linker between FokI domain and dCas9 domain. The sequences of examples of these proteins are provided in SEQ ID NOS: 2 and 18-23. The V5 and Flag tags are not required for these fusion protein function.

FIGS. 5A-5C show sgRNA pair orientation. FIG. 5 A shows schematic models of two types of sgRNA pair orientations. In the PAM-outside orientation, the two PAM sites are outside of the two sgRNA target sites, whereas in the PAM-inside orientation, the two PAM sites are inside the two sgRNA target sites. The spacer is the DNA between two sgRNA target sites (PAM-outside orientation) or between the two PAM sites (PAM-inside orientation). FIG. 5B shows the sgRNA pairs used in the Example 2. FIG. 5C shows an examples of a mouse Rosa26 sgRNA pair. The DNA sequence listed in the figure is a partial mouse Rosa26 locus sequence (chr6:113075997-113076061). The sequences of the two sgRNA are provided in SEQ ID NOS: 32 and 33.

FIGS. 6A-6D show FokI-dCas9 system-mediated mouse genome modifications in mouse Rosa26 locus. FIG. 6A-6C show Surveyor Cel-1 assay results of Rosa26 mutations in Neuro2a cells induced by wild type Cas9 and FokI-dCas9 variants with different pairs of sgRNAs. FIG. 6 D shows sequence alignment of the mutations in mouse Rosa26 locus mediated by a FokI-dCas9 system.

FIGS. 7A, 7C, and 7D show examples of FokI-dCas9 system mediated mutations in human cells and Surveyor Cel-1 assay results of FokI-dCas9 dimer induced target site mutations in human EMX1 gene locus in HEK293 cells. FIG. 7B shows sequence alignment of the EMX1 gene mutations mediated by FokI-dCas9 (L18).

FIGS. 8A-D shows the high specificity of FokI-dCas9 mediated genome mutations. FIGS. 8A and 8B show Surveyor Cel-1 assay results of FokI-dCas9 induced mutations in Rosa26 and human EMX1 gene loci, respectively. FIGS. 8C and 8D show the effects of mismatches in one or both sgRNA's protospacer sequences on the FokI-dCas9 induced mutation efficiency.

FIGS. 9A-B show an application of a FokI-dCas9 system in targeted integration. FIG. 9A shows the targeting strategy and an olio DNA donor used in the test. This donor has an insert of 24 nt comprising a T7 promoter and a BamHI site sequence and has two homology arms (HA-L and HA-R), each with 65 bp. The olio DNA donors sequence is provided in SEQ ID NO: 40. FIG. 9B shows the relative targeted integration efficiency induced by Cas9, FokI-dCas9 and Cas9 nickase (D10A).

FIG. 10 shows efficient genome modifications in mouse embryos mediated by a FokI-dCas9 system.

FIG. 11 shows FokI-dCas9 and ZFN heterodimer induced genome modifications, and targeted integration in mouse Rosa26 locus in Neuro2a cells.

FIG. 12 shows Surveyor Cel-1 assay results of FokI-dCas9 and ZFN heterodimer induced gene mutations in Rosa26 locus in mouse embryos.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF THE PREFERRED EMBODIMENT(S)

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

In accordance with the present disclosure, novel chimeric fusion proteins, polynucleotides, DNA clones, nucleic acids, vectors, and transformed cells, which are useful in the preparation of such chimeric fusion proteins are described. These novel chimeric fusion proteins are useful in methods for genome editing. More particularly, the present disclosure is directed towards chimeric fusion proteins including a DNA modifying domain fused to a catalytically inactive CRISPR associated domain and methods for genome editing using the fusion proteins.

The term “inCas” and “dCas” as used herein refer to a catalytically inactive CRISPR associated protein with active site mutations, for example, the mutations in both RuvC and HNH active sites. For example, the term “inCas9” and “dCas9” as used herein refer to a catalytically inactive Cas9 protein with active site mutations, for example, the mutations in both RuvC and HNH active sites. The dCas or dCas9 also refers to a protein fragments derived from a catalytically inactive Cas9 protein.

As used herein, the term “operably linked” refers to functional linkage between molecules to provide a desired function. For example, “operably linked” in the context of nucleic acids refers to a functional linkage between nucleic acids to provide a desired function such as transcription, translation, and the like, e.g., a functional linkage between a nucleic acid expression control sequence (such as a promoter, signal sequence, or array of transcription factor binding sites) and a second polynucleotide, wherein the expression control sequence affects transcription and/or translation of the second polynucleotide.

As used herein “fused”, “fused to”, “coupled”, “coupled to” and “coupled with” are used interchangeably herein in the context of a polypeptide to refer to a functional linkage between amino acid sequences (e.g., of different domains) such that the polypeptides are part of a single, continuous chain of amino acids that does not occur in nature.

The terms “polypeptide” and “protein” are used interchangeably herein and indicate a molecular chain of amino acids linked through covalent and/or noncovalent bonds. The terms do not refer to a specific length of the product. Thus, peptides and oligopeptides are included within the meaning. The terms include post-expression modifications of the polypeptide, for example, glycosylations, acetylations, phosphorylations and the like. In addition, protein fragments, analogs, mutated or variant proteins, and the like are included within the meaning.

The terms “encoded by”, “encoding” and “encode” as used herein refers to a nucleic acid sequence that codes for a polypeptide sequence. Thus, a suitable “polypeptide,” “protein,” or “amino acid” sequence as used herein may be at least about 60% similar, at least about 70% similar, at least about 80% similar, at least about 90% similar, at least about 95% similar, at least about 96% similar, at least about 97% similar, at least about 98% similar, and at least about 99% similar to a particular polypeptide or amino acid sequence specified below.

The terms “polynucleotide” and “nucleic acid” are used interchangeably herein to refer to a polymeric form of nucleotides of any length, either ribonucleotides (ribonucleic acids) or deoxyribonucleotides (deoxyribonucleic acids). This term refers only to the primary structure of the molecule. Thus, the term includes double-strand DNA and single-stranded DNA as well as double-strand RNA and single-stranded RNA. The term as used herein also includes modifications, such as methylation or capping, and unmodified forms of the polynucleotide.

As used herein a “vector” refers to a replicon to which another polynucleotide segment is attached, such as to bring about the transcription, replication and/or expression of the attached polynucleotide segment. As such, the vector can include origin of replications, promoters, multicloning sites, selectable markers and combinations thereof. Vectors can include, for example, plasmids, viral vectors, cosmids, and artificial chromosomes.

The term “control sequence” as used herein refers to polynucleotide sequences that are necessary to effect the expression of coding sequences to which they are ligated. The nature of such control sequences can differ depending upon the host organism. In prokaryotes, such control sequences may generally include, for example, promoters, ribosomal binding sites and terminators. In eukaryotes, such control sequences may generally include, for example, promoters, terminators and, in some instances, enhancers. The term “control sequence” is thus intended to include at a minimum all components whose presence is necessary for expression, and also may include additional components whose presence is advantageous, for example, leader sequences.

The terms “recombinant polypeptide” or “recombinant protein”, are used interchangeably herein to describe a polypeptide, which by virtue of its origin or manipulation, may not be associated with all or a portion of the polypeptide with which it is associated in nature and/or is fused to a polypeptide other than that to which it is fused in nature. A recombinant polypeptide or protein may not necessarily be translated from a designated nucleic acid sequence. For example, the recombinant polypeptide or protein may also be generated in any manner such as, for example, chemical synthesis or expression of a recombinant expression system.

The terms “recombinant host cells”, “host cells”, “cells”, “cell lines”, “cell cultures”, and other such terms denoting microorganisms or higher eukaryotic cell lines cultured as unicellular entities refer to cells that may be, or have been, used as recipients for transferred nucleic acids and recombinant vectors, and include the original progeny of the original cell that has been transfected.

The term “transformation” and “transfection” as used herein refer to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

As used herein, the term “isolated” refers to polypeptides and polynucleotides that are relatively purified with respect to other bacterial, viral or cellular components that may normally be present in situ, up to and including a substantially pure preparation of the protein and the polynucleotide.

Chimeric Fusion Proteins

In one aspect, the present disclosure is directed to a chimeric fusion protein including a DNA modifying domain fused to a catalytically inactive CRISPR associated protein (dCas) domain. The catalytically inactive CRISPR associated (dCas) domain of the chimeric fusion protein can be obtained, for example, by introducing mutations such as, for example, amino acid substitutions, deletions and insertions, that abolish the Cas protein nuclease activity while retaining its DNA binding activity.

Suitable dCas domains can be obtained from a Cas system. The Cas can be a type I, a type II or a type III system. Non-limiting examples of suitable dCas domains can be from Cas1, Cas2, Cas3, Cas4, Cas5, Cash, Cas7, Cas8 and Cas10, for example. A particularly suitable dCas domain can be a dCas9. The dCas9 can be obtained, for example, by introducing point mutations and/or deletions in the Cas9 protein at both the RuvC and HNH protein active sites (see, Jinek et al., Science 2012; 337:816-821). Introducing two point mutations at the RuvC and HNH active sites abolishes the Cas9 nuclease activity while retaining the Cas9 sgRNA and DNA binding activity. In particular, the two point mutations within the RuvC and HNH active sites can be, for example, Asp10Ala and His840Ala mutations or Asp10Gly and His840Gly mutations of the Cas9 protein from Streptococcus pyogenes (S. pyogenes). Alternatively, Asp10 and His840 of the Cas9 protein from S. pyogenes can be deleted to abolish the Cas9 nuclease activity while retaining its sgRNA and DNA binding activity. Similar mutations can also apply to any other Cas9 proteins from any other nature sources and from any artificially mutated Cas9 proteins. Catalytically inactive Cas9 proteins can also be obtained by point mutations and/or deletions in the RuvC and HNH active sites from any other species such as, for example, Streptococcus thermophiles, Streptococcus salivarius, Streptococcus pasteurianus, Streptococcus mutans, Streptococcus mitis, Streptococcus infantarius, Streptococcus intermedius, Streptococcus equ, Streptococcus agalactiae, Streptococcus anginosus, Bacillus thuringiensis. Finitimus, Streptococcus dysgalactiae, Streptococcus gallolyticus, Streptococcus macedonicus, Streptococcus gordonii, Streptococcus suis, Streptococcus iniae, Neisseria meningitides, Lactobacillus casei, Lactobacillus salivarius, Listeria innocua, Listeria monocytogenes, Lactobacillus buchneri, Lactobacillus paracasei, Lactobacillus sanfranciscensis, Lactobacillus fermentum, Listeria innocua serovar, Lactobacillus rhamnosus, Lactobacillus casei, Lactobacillus sanfranciscensis, Haemophilus sputorum, Geobacillus, Enterococcus hirae, Enterococcus faecalis, Bacillus cereus, Treponema socranskii, Finegoldia magna and others. Similar catalytically inactive mutations can also apply to any other Cas9 proteins from any other natural sources, from any artificially mutated Cas9 proteins, and/or from any artificially created protein fragments that comprise a dCas9 like sgRNA binding domain.

The DNA modifying domain of the chimeric fusion protein can be any DNA modification enzyme known to those skilled in the art. The DNA modifying domain of the chimeric fusion protein can be a full-length DNA modifying enzyme. The DNA modifying domain of the chimeric fusion protein can also be a domain obtained from the full-length DNA modifying enzyme in which the domain retains the DNA modifying activity of the full-length DNA modifying enzyme. A particularly suitable domain of a DNA modifying enzyme can be any catalytic domain of the DNA modifying enzyme. Particularly suitable DNA modifying domains can be those that require dimerization or protein/domain complementation to reconstitute their catalytic activities.

Suitable DNA modifying domains can be, for example, an endonuclease, an exonuclease, a DNA methyltransferase, a DNA glycosidase, a DNA polymerase, a DNA ligase, a DNA topoisomerase, a DNA kinase, an oxidoreductase, and a histone deacetylase.

Suitable DNA modifying domains can be, for example, any endonuclease known by those skilled in the art. Particularly suitable DNA modifying domain can be, for example, type II restriction endonucleases including, for example, type IIS restriction endonucleases. A particularly suitable type IIS restriction endonuclease can be FokI and an endonuclease domain obtained from FokI. The activity of the FoKI endonuclease domain relies on dimerization. Other suitable type IIS restriction endonucleases can be, for example, AlwI, BsmFI, BspCNI, BtsCI, HgaI, eco571R, mboIIR, begIB, and/or any Type IIS restriction enzymes, including, but not limited to, those listed in New England Biolabs' websites under the group of ‘Type IIS” enzymes (www.neb.com/tools-and-resources/interactive-tools/enzyme-finder?searchType-6).

Particularly suitable DNA methyltransferases can be, for example, a mammalian DNA methyltransferase (e.g., DNMT1, DNMT3A, and DNMT), an N-6 adenine-specific DNA methylase, an N-4 cytosine-specific DNA methylase, a C-5 cytosine-specific DNA methylase and/or any other methyltransferases.

The above fusion proteins can be produced by expression of polynucleotides encoding the same. These too permit a degree of variability in their sequence, as for example due to degeneracy of the genetic code, codon bias in favor of the host cell expressing the polypeptide, and conservative amino acid substitutions in the resulting protein. Consequently, the fusion proteins and constructs of the present disclosure include not only those which are identical in sequence to the above described fusion protein but also those variant polypeptides with the structural and functional characteristics that remain substantially the same. Such variants (or “analogs”) may have a sequence homology (“identity”) of 60%, 70%, 80%, 90%, 95%, 96%, 97%, 98%, 99% or more with the sequences described herein. In this sense, techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity. A so-termed “percent similarity” may then be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded therein, and comparing this to a second amino acid sequence. In general, “identity” refers to an exact nucleotide to nucleotide or amino acid to amino acid correspondence of two polynucleotides or polypeptide sequences, respectively. Two or more polynucleotide sequences can be compared by determining their “percent identity”, as can two or more amino acid sequences. The programs available in the Wisconsin Sequence Analysis Package, Version 8 (available from Genetics Computer Group, Madison, Wis.), for example, the GAP program, are capable of calculating both the identity between two polynucleotides and the identity and similarity between two polypeptide sequences, respectively. Other programs for calculating identity or similarity between sequences are known by those skilled in the art.

Linkers

The chimeric fusion protein can further include at least one linker. The length of the linker in the chimeric fusion protein can be adjusted to fit different length of spacer (gap) sequence between two sgRNA binding sites as described herein. Different linkers are suitable for different spacer lengths. The spacer sequence length can vary, but can be from about 1 nucleotides to about 50 nucleotides (nt). Non-limiting examples of particularly suitable spacer length can be from 13 nucleotides to 23 nucleotides and 30 nucleotides. Those skilled in the art can readily determine the length of the linker such that a sufficient number of amino acids are included to allow the DNA modifying domains of the chimeric fusion protein monomers to form a dimer. Suitable linkers can be any amino acids as determined by those skilled in the art. Suitable linkers can be 1 amino acid (aa), 2aa, 3aa, 4aa, 5aa, baa, 7aa, Baa, 9aa, 10aa, 11aa, 12aa, 13aa, 14aa, 15aa, 16aa, 17aa, 18aa, 19aa or 20aa. Non-limiting examples of particularly suitable linkers can be, for example, a Linker L4, Linker L5, Linker L8, Linker L18 and Linker 40 (SEQ ID NOS: 25-29) or those of SEQ ID NOS: 4-5.

Nuclear Localization Signal Sequences

The chimeric fusion protein can further include at least one nuclear localization signal sequence (NLS). The NLS is an amino acid sequence which results in the importation of the chimeric fusion protein into the cell nucleus by nuclear transport. The NLS can be, for example, one or more short sequences of positively charged lysines or arginines exposed on the protein surface; can be either monopartite or bipartite; can be either classical or nonclassical NLSs. Suitable NLSs can be, for example, a PY-NLS motif; PKKKRKV (SEQ ID NO:6); the acidic M9 domain of hnRNP A1, the sequence KIPIK (SEQ ID NO:7) of the yeast transcription repressor Matα2, the complex signals of U snRNPs, the RKRRR (SEQ ID NO:14) motif from Notch1 protein, the KRKRK (SEQ ID NO:15) from Notch 2 protein, the RRKR (SEQ ID NO:16) motif from Notch3 protein, the RRRRR (SEQ ID NO: 17) motif from Notch4 protein, and any other NLSs from any nuclear proteins known or later discovered by those skilled in the art.

The chimeric fusion protein can further include at least one linker and at least one nuclear localization signal sequence. Suitable linkers and nuclear localization signal sequences are described herein.

The domain structure of the DNA modifying enzyme-dCas domain can be in a variety of orientations. In one embodiment, for example, the dCas domain can be located at the C-terminus of the fusion protein such that the chimeric fusion protein is oriented from N-terminus to C-terminus as: DNA modifying domain-dCas domain. In another embodiment, for example, the dCas domain can be located at the N-terminus of the fusion protein such that the chimeric fusion protein is oriented from N-terminus to C-terminus as: dCas domain-DNA modifying domain.

Particularly suitable orientation of the chimeric protein is that dCas domain is located at the C-terminus of the fusion protein such that the chimeric fusion protein is oriented from N-terminus to C-terminus as: DNA modifying domain-dCas domain.

The domain structure of the DNA modifying domain-Linker-dCas domain can also be in a variety of orientations. In one embodiment, for example, the dCas domain can be located at the C-terminus of the fusion protein such that the chimeric fusion protein is oriented from N-terminus to C-terminus as: DNA modifying domain-Linker dCas domain. In another embodiment, for example, the dCas domain can be located at the N-terminus of the fusion protein such that the chimeric fusion protein is oriented from N-terminus to C-terminus as: dCas domain-Linker-DNA modifying domain.

Particularly suitable orientation of the chimeric protein is that dCas domain is located at the C-terminus of the fusion protein such that the chimeric fusion protein is oriented from N-terminus to C-terminus as: DNA modifying domain-Linker-dCas domain. The domain structure of the NLS-DNA modifying domain-Linker-dCas domain can also be in a variety of orientations. In one embodiment, for example, the NLS can be located at the N-terminus of the fusion protein such that the chimeric fusion protein is oriented from N-terminus to C-terminus as: NLS-DNA modifying domain-Linker-dCas domain. In another embodiment, for example, the NLS can be located at the C-terminus of the fusion protein such that the chimeric fusion protein is oriented from N-terminus to C-terminus as: DNA modifying domain-dCas domain-NLS. In another embodiment, for example, the NLS can be located between the dCas domain and DNA modifying domain of the fusion protein such that the chimeric fusion protein is oriented from N-terminus to C-terminus as: DNA modifying domain-Linker-NLS-dCas9.

The domain structure of the NLS-DNA modifying domain-Linker-dCas domain can also be in a variety of orientations. In one embodiment, for example, the NLS can be located at the N-terminus of the fusion protein such that the chimeric fusion protein is oriented from N-terminus to C-terminus as: NLS-DNA modifying domain-Linker-dCas domain. In another embodiment, for example, the NLS can be located at the C-terminus of the fusion protein such that the chimeric fusion protein is oriented from N-terminus to C-terminus as: DNA modifying domain-Linker-dCas domain-NLS. In one embodiment, for example, the NLS can be located between the dCas domain and linker such that the chimeric fusion protein is oriented from N-terminus to C-terminus as: DNA modifying domain-NLS-Linker-dCas domain. In one embodiment, for example, the NLS can be located between the DNA modifying domain and linker such that the chimeric fusion protein is oriented from N-terminus to C-terminus as: DNA modifying domain-NLS-Linker-dCas domain.

In another embodiment, the chimeric fusion protein can include two NLS's in which the domain structure of the DNA modifying domain-Linker-dCas domain including two NLS's can be in a variety of orientations. In one embodiment, for example, one NLS can be located at the N-terminus and one can be located at the C-terminus such that the chimeric fusion protein is oriented from N-terminus to C-terminus as: NLS-DNA modifying domain-Linker-dCas domain-NLS. In one embodiment, for example, one NLS can be located at the N-terminus or C-terminus and the second NLS can be located between the dCas domain and the linker, between the linker and DNA modifying domain such that the chimeric fusion protein is oriented from N-terminus to C-terminus as: NLS-DNA modifying domain-linker-NLS-dCas domain; NLS-DNA modifying domain-NLS-Linker-dCas domain; DNA modifying domain-linker-NLS-dCas domain-NLS; DNA modifying domain-NLS-Linker-dCas domain-NLS.

In another embodiment, the chimeric fusion protein can include two or more linkers and two or more NLS's in which the domain structure of the chimeric fusion protein including the two or more linkers and the two or more NLS's can be in a variety of orientations. In one embodiment, for example, one NLS can be located at the N-terminus and one can be located at the C-terminus such that the chimeric fusion protein is oriented from N-terminus to C-terminus as: NLS-Linker-DNA modifying domain-Linker-dCas-NLS, NLS DNA modifying domain-Linker-dCas-NLS, NLS-DNA modifying domain-Linker-dCas-linker-NLS, and NLS-Linker-NLS-DNA modifying domain-Linker-dCas.

FokI-dCas9 Fusion Proteins

In another aspect, the present disclosure is directed to a chimeric fusion protein having a dCas9 domain fused to a FokI domain. The dCas9 domain of the chimeric fusion protein can be obtained, for example, by introducing point mutations in the Cas9 protein as described herein. In particular, the dCas9 can be a dCas9 having two point mutations within the RuvC and HNH active sites such as, for example, Asp10Ala and His840Ala mutations and Asp10Gly and His840Gly mutations, and deletions of Asp10 and His840 of the Cas9 from S. pyogenes. Catalytically inactive Cas9 proteins can also be obtained from any other species such as, for example, Streptococcus thermophiles, Streptococcus salivarius, Streptococcus pasteurianus, Streptococcus mutans, Streptococcus mitis, Streptococcus infantarius, Streptococcus intermedius, Streptococcus equ, Streptococcus agalactiae, Streptococcus anginosus, Bacillus thuringiensis. Finitimus, Streptococcus dysgalactiae, Streptococcus gallolyticus, Streptococcus macedonicus, Streptococcus gordonii, Streptococcus suis, Streptococcus iniae, Neisseria meningitides, Lactobacillus casei, Lactobacillus salivarius, Listeria innocua, Listeria monocytogenes, Lactobacillus buchneri, Lactobacillus paracasei, Lactobacillus sanfranciscensis, Lactobacillus fermentum, Listeria innocua serovar, Lactobacillus rhamnosus, Lactobacillus casei, Lactobacillus sanfranciscensis, Haemophilus sputorum, Geobacillus, Enterococcus hirae, Enterococcus faecalis, Bacillus cereus, Treponema socranskii, Finegoldia magna and others Cas9s by point mutations and/or deletions in the RuvC and HNH active sites. Similar catalytically inactive mutations can also apply to any other Cas9 proteins or Cas9 like proteins from any other nature sources and from any artificially mutated Cas9 proteins.

The FokI domain can be, for example, a wild type FokI nuclease catalytic domain, a modified homo monomeric FokI nuclease cleavage domain, a FokI nuclease domain containing the FokI nuclease DNA cleavage domain. The FokI domain can also be obligate heterodimeric FokI domain variants such as, for example, a DD/RR pair, a KK/EL pair, a KKR/ELD pair and other pairs. In these cases, the FokI-dCas9 fusion protein needs to be used in pairs such as, for example, for example, FokI(KKR)-dCas9 pairs with FokI(ELD)-dCas9; FokI(DD)-dCas9 pairs with FokI(RR)-dCas9 and FokI(KK)-dCas9 pairs with FokI(EL)-dCas9. If the FokI domain in the FokI-dCas9 fusion protein are from heterodimeric domain pairs, an equal amount of two different monomeric FokI fusion proteins, each with a corresponding FokI domain, will be introduced together into cells or organisms to further improve cleavage specificity. In another embodiment, the FokI domain can also be one from a catalytically inactive FokI, which in use can be paired with a catalytically active FokI domain to generate a nick in the target DNA.

The chimeric fusion protein having a FokI domain fused to a dCas9 domain can further include at least one linker as described herein. The chimeric fusion protein having a FokI domain fused to a dCas9 domain can further include at least one NLS as described herein. The chimeric fusion protein having a FokI domain fused to a dCas9 domain can further include at least one linker and at least one NLS as described herein.

The preferred N-terminus to C-terminus orientation of the Fok-dCas9 fusion protein is the FokI-Linker-dCas9-NLS, NLS-FokI-Linker-dCas9, or NLS-FokI-Linker-dCas9-NLS. The preferred structure is the FokI-domain fused at the N-terminus of dCas9 domain. A linker may be included between NLS and FokI domain if the NLS is fused to the N-terminus of FokI-dCas9 fusion protein.

In another aspect, the present disclosure is directed to an isolated nucleic acid that includes a nucleotide sequence encoding a chimeric fusion protein including a DNA modifying domain fused to a dCas domain. Suitable chimeric fusion proteins can include dCas domains, DNA modifying domains, linkers and nuclear localization signal sequences as described herein. A particularly suitable dCas domain can be a dCas9 domain as described herein. A particularly suitable DNA modifying domain can be a FokI domain as described herein. The isolated nucleic acid can further include a nucleotide sequences encoding linkers and NLSs as described herein. The nucleic acid can be, for example, a DNA, a DNA fragment, a RNA, a RNA fragment, and a DNA plasmid.

In another aspect, the present disclosure is directed to a vector including a nucleic acid sequence encoding a chimeric fusion protein including a DNA modifying domain fused to a catalytically inactive Cas (dCas) domain. Suitable chimeric fusion proteins can include dCas proteins, DNA modifying enzymes, linkers and NLSs as described herein. A particularly suitable dCas domain can be a dCas9 domain as described herein. A particularly suitable DNA modifying domain can be a FokI domain as described herein. The vector can further include linkers and NLSs as described herein.

In another aspect, the present disclosure is directed to a cell including a nucleic acid sequence encoding a chimeric fusion protein including a DNA modifying domain fused to a catalytically inactive Cas (dCas) domain. Suitable chimeric fusion proteins can include dCas proteins, DNA modifying enzymes, linkers and NLSs as described herein. A particularly suitable dCas domain can be a dCas9 domain as described herein. A particularly suitable DNA modifying domain can be a FokI domain as described herein. Suitable cells can be, for example, prokaryotic cells and eukaryotic cells. Suitable prokaryotic cells can be, for example, bacterial cells. Suitable eukaryotic cells can be for example, mammalian cells and plant cells. Suitable mammalian cells can be, for example, human cells, fish cells, Drosophila cells, C. elegans cells, silkworm cells, mouse cells, rat cells, rabbit cells, pig cells, cow cells, cat cells, dog cells, chicken cells, embryos, and other animal and plant cells.

In another aspect, the present disclosure is directed to a cell including a vector including a nucleic acid sequence encoding a chimeric fusion protein including a DNA modifying domain fused to a catalytically inactive Cas (dCas) domain. Suitable chimeric fusion proteins can include dCas proteins, DNA modifying enzymes, linkers and NLSs as described herein. A particularly suitable dCas domain can be a dCas9 domain as described herein. A particularly suitable DNA modifying domain can be a FokI domain as described herein. Suitable cells can be, for example, prokaryotic cells and eukaryotic cells. Suitable prokaryotic cells can be, for example, bacterial cells. Suitable eukaryotic cells can be for example, mammalian cells and plant cells. Suitable mammalian cells can be, for example, human cells, fish cells, Drosophila cells, C. elegans cells, silkworm cells, mouse cells, rat cells, rabbit cells, pig cells, cow cells, cat cells, dog cells, chicken cells, embryos, and other animal and plant cells.

In another aspect, the present disclosure is directed to an organism including a nucleic acid sequence encoding a chimeric fusion protein including a DNA modifying domain fused to a catalytically inactive Cas (dCas) domain. Suitable chimeric fusion proteins can include dCas proteins, DNA modifying enzymes, linkers and NLSs as described herein. A particularly suitable dCas domain can be a dCas9 domain as described herein. A particularly suitable DNA modifying domain can be a FokI domain as described herein. Suitable organisms can be, for example, humans, plants, fish, Drosophila, C. elegans, silkworms, mice, rats, rabbits, pigs, cows, cats, dogs, chickens and other animals.

In another aspect, the present disclosure is directed to an organism including a vector including a nucleic acid sequence encoding a chimeric fusion protein including a DNA modifying domain fused to a catalytically inactive Cas (dCas) domain. Suitable chimeric fusion proteins can include dCas proteins, DNA modifying enzymes, linkers and nuclear localization sequences as described herein. A particularly suitable dCas domain can be a dCas9 domain as described herein. A particularly suitable DNA modifying domain can be a FokI domain as described herein. The vector can further include linkers and NLSs as described herein. Suitable organisms can be, for example, plants, fish, Drosophila, C. elegans, silkworms, mice, rats, rabbits, pigs, cows, cats, dogs, chickens and other animals.

Methods of Gene Editing

In another aspect, the present disclosure is directed to methods of gene editing. The method includes introducing at least two monomeric chimeric fusion proteins into a cell, wherein the at least two monomeric chimeric fusion proteins each comprises a DNA modifying domain fused to a dCas domain fused; introducing a first guide RNA (sgRNA) and a second guide RNA (sgRNA) into the cell, wherein the first sgRNA and the second sgRNA comprise an at least 12-20 nucleotide sequence complementary to two adjacent target DNA nucleotide sequences and wherein the first sgRNA forms a first complex with one chimeric fusion protein monomer and wherein the second sgRNA forms a second complex with one chimeric fusion protein monomer to direct the at least two monomeric chimeric fusion proteins to the adjacent target DNA nucleotide sequences wherein the two monomeric chimeric fusion proteins form a DNA modifying domain dimer and induce a DNA modification in the target DNA.

In another aspect, the present disclosure is directed to methods of gene editing. The method includes introducing at least two monomeric chimeric fusion proteins into an organism, wherein the at least two monomeric chimeric fusion proteins each includes a DNA modifying domain fused to a catalytically inactive Cas (dCas) domain; introducing a first guide RNA (sgRNA) and a second guide RNA (sgRNA) into the organism, wherein the first sgRNA and the second sgRNA comprise an at least 12-20 nucleotide sequence complementary to two adjacent target DNA nucleotide sequences and wherein the first sgRNA forms a first complex with one chimeric fusion protein monomer and wherein the second sgRNA forms a second complex with one chimeric fusion protein monomer to direct the at least two monomeric chimeric fusion proteins to the adjacent target DNA nucleotide sequences wherein the two monomeric chimeric fusion proteins form a DNA modifying domain dimer and induce a DNA modification in the target DNA.

The dCas domain and DNA modifying domain of the chimeric fusion protein can be those described herein. The chimeric fusion protein of the method can further include linkers and NLSs as described herein. The methods also include co-introduction of two different chimeric fusion proteins, the dCas9 can be different and the FokI can also be different.

The chimeric fusion protein can be introduced into the cell or the organism as a protein or as a nucleic acid sequence encoding the chimeric fusion protein. When introduced as a nucleic acid sequence, the chimeric fusion protein is expressed by the cell or the organism. The nucleic acid sequence can be a DNA (with an appropriate promoter and a poly A signal sequence) or mRNA (with Cap and Poly A tail). The chimeric fusion protein can also be introduced as a polypeptide, or protein.

The method also includes introducing guide RNAs (sgRNAs) into the cell or the organism. The guide RNAs (sgRNAs) include nucleotide sequences that are at complementary to two adjacent sequences of the target chromosomal DNA. The sgRNA can be, for example, an engineered single chain guide RNA that comprises a crRNA sequence (complementary to the target DNA sequence) and a common tracrRNA sequence, or as crRNA-tracrRNA hybrids. The sgRNAs can be introduced into the cell or the organism as a DNA (with an appropriate promoter), as an in vitro transcribed RNA, or as a synthesized RNA.

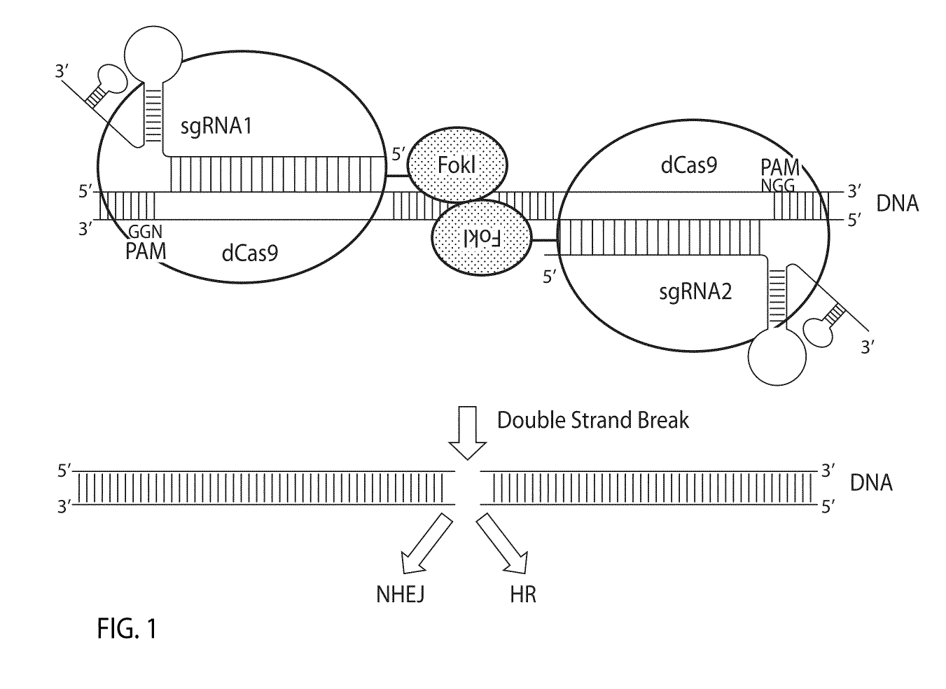

The preferred orientation of the two sgRNAs in a pair is that the two PAM sites of the sgRNAs are located outside of the two sgRNA target site as illustrated in the FIG. 1.

The suitable spacer length between the two sgRNAs is between 1 to 50 nucleotides. Non limiting examples of suitable spacer is between 13 and 23, and a 30 nucleotides. Non-limiting examples of most suitable spacer is 18, 19, or 30 nucleotides.

The suitable sgRNA has at least 12 nucleotide match to the target DNA sequence.

The chimeric fusion protein, the sgRNAs or both can be introduced into the cell or the organism by standard delivering methods known to those skilled in the art. Suitable delivery methods can be, for example, transfection, electroporation, nucleofection and injection.

The specificity of the binding by the Cas domain to the target DNA is mediated by the sgRNA that mimics the natural crRNA-tracrRNA hybrid. Target DNA recognition and cleavage use a sequence complementarity between the target site and the sgRNA sequence (the crRNA part), as well as a protospacer adjacent motif (PAM). The sequence complementarity between the target site and the sgRNA can be about 12 nucleotides. The sequence complementarity between the target site and the sgRNA can also be about 20 nucleotides. The sequence complementarity between the target site and the sgRNA can also be more than about 12 nucleotides. The sequence complementarity between the target site and the sgRNA can also be more than about 20 nucleotides. The sequence complementarity between the target site and the sgRNA can also be from about 12 nucleotides to about 20 nucleotides. Thus, as a pair, two sgRNAs can target a site of about 24 nucleotides or more, including from about 24 nucleotides to about 40 nucleotides, and even greater than 40 nucleotides. The sequence of the two PAM sites on a target DNA can be the same or different. A PAM sequence can be from about 2 to about 4 nucleotides, for example. Suitable PAM sequences can be, for example, the 3-nucleotide NGG sequence from S. pyogenes Cas9 and the 3-nucleotide NAG sequence from S. pyogenes Cas9. Cas proteins from different sources can have different PAM sequences. If two monomeric chimeric fusion proteins are created using different Cas domains with different PAM sequences, an equal amount of the two different chimeric fusion proteins (each with its own dCas domain), together with two corresponding sgRNAs can be introduced into cells or organisms. For example, Cas9 proteins from different sources can have different PAM sequences, and thus, if two monomeric chimeric fusion proteins are created using different Cas9 domains that use different PAM sequences, an equal amount of the two different chimeric fusion proteins (each with its own dCas9 domain), together with two corresponding sgRNAs can be introduced into the cell or the organism.

The guide RNA (sgRNA) can include, for example, a nucleotide sequence that comprises an at least 12-20 nucleotide sequence complementary to the target DNA sequence and can include a common scaffold RNA sequence at its 3′ end. As used herein, “a common scaffold RNA” refers to any RNA sequence that mimics the tracrRNA sequence or any RNA sequences that function as a tracrRNA. As described herein, the sequence complementarity between the target DNA site and the sgRNA can be about 12 nucleotides. The sequence complementarity between the target DNA site and the sgRNA can also be about 20 nucleotides. The sequence complementarity between the target DNA site and the sgRNA can also be more than about 12 nucleotides. The sequence complementarity between the target DNA site and the sgRNA can also be more than about 20 nucleotides. The sequence complementarity between the target DNA site and the sgRNA can also be from about 12 nucleotides to about 20 nucleotides. An example of a particularly suitable common scaffold RNA (equivalent to a tracrRNA) sequence is SEQ ID NO: 3, but other scaffold RNAs can also be used in the present disclosure. A sgRNA sequence can be determined, for example, by identifying a sgRNA binding site by locating a PAM sequence in the target DNA, and then choosing about 12 nucleotides to about 20 or more nucleotides immediately upstream of the PAM site. For Cas9 from S. pyogenes, for example, its PAM sequence can be, for example, NGG or NAG downstream of the 3′ end of an sgRNA target site. For chimeric fusion proteins that dimerize for DNA modifying domain activity, two sgRNAs (e.g., sgRNA1 and sgRNA2) can be used to guide each monomeric chimeric fusion protein to each site of the target DNA. The two sgRNA binding sites are in adjacent regions, and preferably on the different strands of a target DNA. For chimeric fusion proteins that dimerize for activity, the two sgRNA target sites should be close so that the DNA modifying enzyme can be in close proximity, but not overlap. The spacer sequence (gap size) between the two sgRNA binding sites on a target DNA can depend on the target DNA sequence and can be determined by those skilled in the art. In particular, the gap size can be, for example, 1 nucleotide. The gap size can also be more than 1 nucleotide. The gap size can also be from about 1 nucleotide to about 50 nucleotides. The examples of preferred gap (Spacer) length is between 13 and 23 nucleotides, and a 30 nucleotides. From the gap size, the length of the linker in the chimeric fusion protein can also be determined.

The preferred orientation of the 2 sgRNAs in a pairs should be that the 2 PAM sites of the 2sgRNAs are located outside of the 2 sgRNA binding sites, as illustrated in FIG. 1.

The DNA binding specificity of the chimeric fusion protein depends on the DNA binding specificity of the dCas domain, which depends on the sequence of the sgRNA, and the DNA modifying domain activity of the chimeric fusion protein depends on the DNA modifying domain. In applications where the DNA modifying domain of the chimeric fusion protein functions as a dimer, monomeric forms of the chimeric fusion protein does not cleave the target DNA, even in the presence of an sgRNA. When a pair of two different sgRNAs targeting two adjacent sites on a double strand DNA is present, two monomeric chimeric fusion proteins can bind to the two close adjacent sites on the target DNA, which leads to the dimerization of the two DNA modifying domains that can induce a DNA modification in the target DNA. For example, a dimer of two DNA modifying domains having endonuclease activity can cleave the target DNA sequence between the two sgRNA target sites.

Suitable cells can be, for example, prokaryotic cells and eukaryotic cells. Suitable prokaryotic cells can be, for example, bacterial cells. Suitable eukaryotic cells can be for example, animal cells, plant cells, and human cells. Suitable animal cells can be, for example, fish cells, Drosophila cells, C. elegans cells, silkworm cells, mouse cells, rat cells, rabbit cells, pig cells, cow cells, cat cells, dog cells, chicken cells, embryos, and other animal cells. Suitable organisms can be, for example, plants, fish, Drosophila, C. elegans, silkworms, mice, rats, rabbits, pigs, cows, cats, dogs, chickens and other animals.

The target DNA can be chromosomal DNA and plasmid DNA.

The DNA modification to the target DNA can be, for example, a double-strand break, a single-strand nick to the target DNA, a methylation, and a demethylation.

The method can further include introducing a genetic modification in the target DNA. The genetic modification can be any genetic modification known to those skilled in the art. When co-introducing a donor DNA, suitable genetic modifications can be, for example, a DNA deletion, a gene disruption, a DNA insertion, a DNA inversion, a point mutation, a DNA replacement, a knock-in, a knock-out, a knock-down and other genetic modifications in the target DNA at the site of a double-strand break or the single-stranded nick.

Methods of Gene Editing Using a FokI-dCas9 Fusion Protein

In another aspect, the present disclosure is directed to a method of inducing double-strand breaks in a target DNA. The method includes introducing at least two FokI-dCas9 fusion protein monomers into a cell; introducing a first guide RNA (sgRNA) and a second guide RNA (sgRNA) into the cell, wherein the at least two sgRNAs comprise an at least 12-20 nucleotide sequence complementary to at least two target DNA nucleotide sequences and wherein the first sgRNA forms a first complex with one FokI-dCas9 fusion protein monomer and wherein the second sgRNA forms a second complex with one FokI-dCas9 fusion protein monomer to direct the at least two FokI-dCas9 fusion protein monomers to adjacent sites of the target DNA, wherein the at least two FokI-dCas9 fusion protein monomers form a FokI dimer and induce DNA double-strand breaks in the target DNA.

The FokI-dCas9 fusion protein monomers can be introduced into the cell as a polypeptide, or a protein. Alternatively, the FokI-dCas9 fusion protein monomers can introduced into the cell as a nucleic acid sequence that encodes the FokI-dCas9 fusion protein monomers.

In another aspect, the present disclosure is directed to a method of inducing double-strand breaks in a target DNA. The method includes introducing at least two FokI-dCas9 fusion protein monomers into a cell; introducing a first guide RNA (sgRNA) and a second guide RNA (sgRNA) into the cell, wherein the at least two sgRNAs comprise an at least 12-20 nucleotide sequence complementary to at least two target DNA nucleotide sequences and wherein the first sgRNA forms a first complex with one chimeric fusion protein monomer and wherein the second sgRNA forms a second complex with one chimeric fusion protein monomer to direct the at least two FokI-dCas9 fusion protein monomers to adjacent sites of the target DNA, wherein the at least two FokI-dCas9 fusion protein monomers form a FokI dimer and induce DNA double-strand breaks in the target DNA.

The FokI-dCas9 fusion protein monomers can be introduced into the organism as polypeptides. Alternatively, the FokI-dCas9 fusion protein monomers can introduced into the organism as a nucleic acid sequence that encodes the FokI-dCas9 fusion protein monomers.

The FokI-dCas9 fusion protein monomers can further include linkers and NLSs as described herein. Suitable dCas9 domains, linkers and NLSs as described herein. A particularly suitable dCas domain can be a dCas9 domain as described herein.

As FokI only cleaves DNA as a dimer, a monomeric FokI-dCas9 fusion protein does not cleave DNA, even in the presence of one type of sgRNA. When a pair of sgRNAs targeting two adjacent sites on a double strand DNA is present, two monomeric FokI-dCas9 fusion proteins can bind to the two adjacent sites on the target DNA, which leads to the dimerization of the two FokI domains. The dimerized FokI domains can then cleave the target DNA and induce a DNA double-strand breaks in the target DNA. Cleavage can occur between the two sgRNA target sites. The double-strand breaks (DSBs) induced by the FokI-dCas9 dimer (in the presence of two sgRNAs) can be repaired by, for example, error-prone nonhomologous end joining (NHEJ) or homologous recombination (HR) to mediate genetic modifications.

The method can further include introducing a genetic modification in the target DNA. The genetic modification can be any genetic modification known to those skilled in the art. Suitable genetic modifications can be, for example, a DNA deletion, a gene disruption, an insertion, an inversion, a point mutation, a DNA replacement, a knock-in, a knock-out, a knock-down and other genetic modifications in the target DNA at the site of a double-strand break or a single-strand nick.

Methods of Gene Editing Using Chimeric Fusion Proteins Paired with a Nuclease

In another aspect, the present disclosure is directed to a method of gene editing. The method includes introducing a chimeric fusion protein monomer that comprises a FokI domain fused to a dCas9 domain into a cell or an organism; introducing a guide RNA (sgRNA) into the cell or the organism, wherein the sgRNA comprises an at least 12-20 nucleotide sequence complementary to a sequence in a target DNA and wherein the sgRNA forms a complex with the chimeric fusion protein monomer; wherein the sgRNA guides binding of the chimeric fusion protein monomer to the target DNA; and introducing a different nuclease into the cell or the organism, wherein the nuclease comprises a FokI domain; wherein the FokI domain of the chimeric fusion protein monomer and the FokI domain of the nuclease form a FokI dimer and induces double-strand breaks in the target DNA.

The sgRNA guides binding of the chimeric fusion protein monomer to the target DNA. Thus, the sgRNA and chimeric fusion protein monomer forms a complex at the target DNA. The different nuclease, via its DNA-binding domain as described herein, is designed to bind to a site in the target DNA sequence such that the nuclease is positioned adjacent to the chimeric fusion protein monomer. This allows the DNA modifying domain of the chimeric fusion protein monomer and the DNA-cleaving domain of the nuclease to form a dimer, which can then induce double-strand breaks or single-strand nicks in the target DNA.

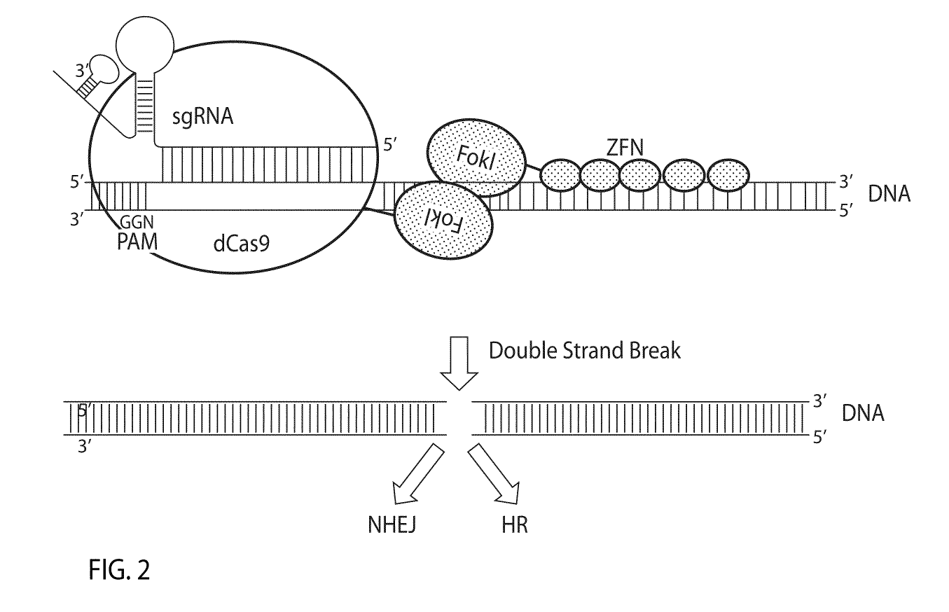

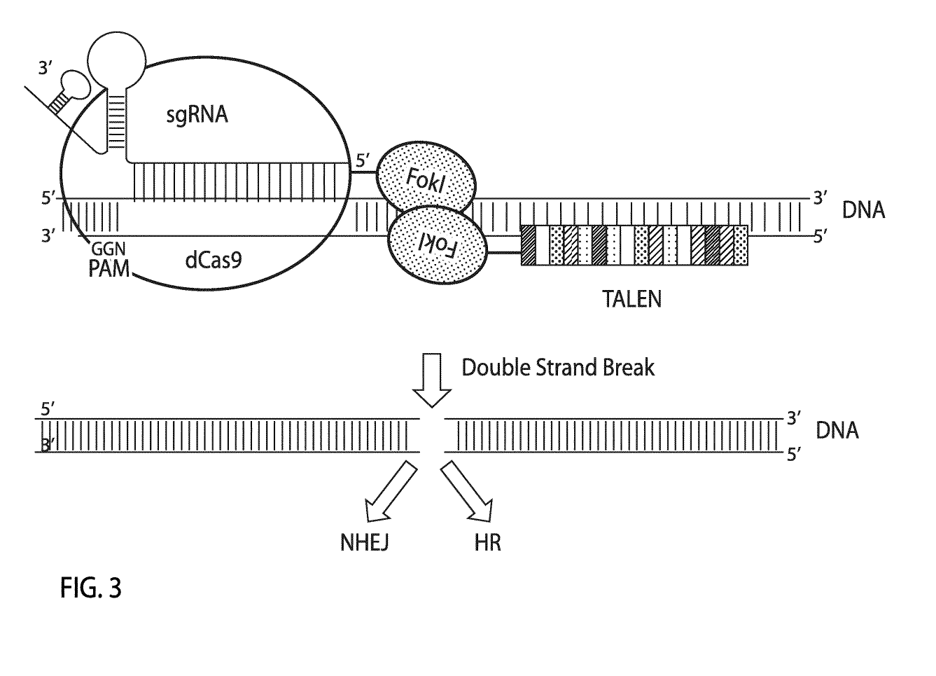

The preferred sgRNA orientation in this FokI-dCas9 and nuclease heterodimer is that the PAM site of the sgRNA is located outside of the sgRNA and the nuclease target sites, as illustrated in FIGS. 2 and 3.

The DNA modification to the target DNA can be, for example, a double-strand break or a single-strand nick to the target DNA.

The chimeric fusion protein can further include linkers and NLSs as described herein. Suitable dCas domains, DNA modifying domains, linkers and NLSs are described herein. A particularly suitable dCas domain can be a dCas9 domain as described herein. A particularly suitable DNA modifying domain can be FokI as described herein.

Suitable nucleases can be, for example, a Zinc Finger Nuclease (ZFN) and Transcription Activator Like Effector Nuclease (TALEN). Suitable ZFNs and TALENs include a DNA-binding domain and a DNA-cleaving domain. Particularly suitable DNA-cleaving domains can be, for example, type IIS restriction endonucleases as described herein. A particularly suitable DNA-cleaving domain can be FokI as described herein. The FIG. 2 illustrates the FokI-dCas9 and ZFN heterodimer mediated DNA double strand break. The FIG. 3 illustrates the FokI-dCas9 and TALEN heterodimer mediated DNA double strand break.

The DNA-binding domain of a ZFN can be, for example, zinc finger repeats. The number of zinc finger repeats can be from about 3 to about 6. The DNA-binding domain of a TALEN can be a TAL (transcription activator-like) effector DNA binding domain.

The method can further include introducing a genetic modification in the target DNA. The genetic modification can be any genetic modification known to those skilled in the art. Suitable genetic modifications can be, for example, a DNA deletion, a gene disruption, a DNA insertion, a DNA inversion, a point mutation, a DNA replacement, a knock-in, a knock-out, a knock-down and other genetic modifications in the target DNA at the site of a double-strand break or a single-strand nick.

Without being bound by theory, the chimeric fusion protein plus sgRNA targets to one site of the target DNA, whereas the nuclease targets to a site of the target DNA that is adjacent to the chimeric fusion protein plus sgRNA. Target DNA modification occurs when the DNA modifying domain of the chimeric fusion protein and the DNA-cleaving domain nuclease are in close proximity such that the domains can dimerize. An advantage of this combination is that some target DNA sequences may be suitable for one kind of binding (either by the chimeric fusion protein/sgRNA or the nuclease) while other target DNA sequences may be suitable for a different kind of binding as determined by their sequence binding requirements.

The disclosure will be more fully understood upon consideration of the following non-limiting Examples.

### EXAMPLES

### Example 1

### Engineering FokI-dCas9 Fusion Protein Encoding DNA Constructs

In this Example, a chimeric fusion protein having a FokI nuclease domain fused to catalytically inactive Cas9 domain (dCas9) is described.

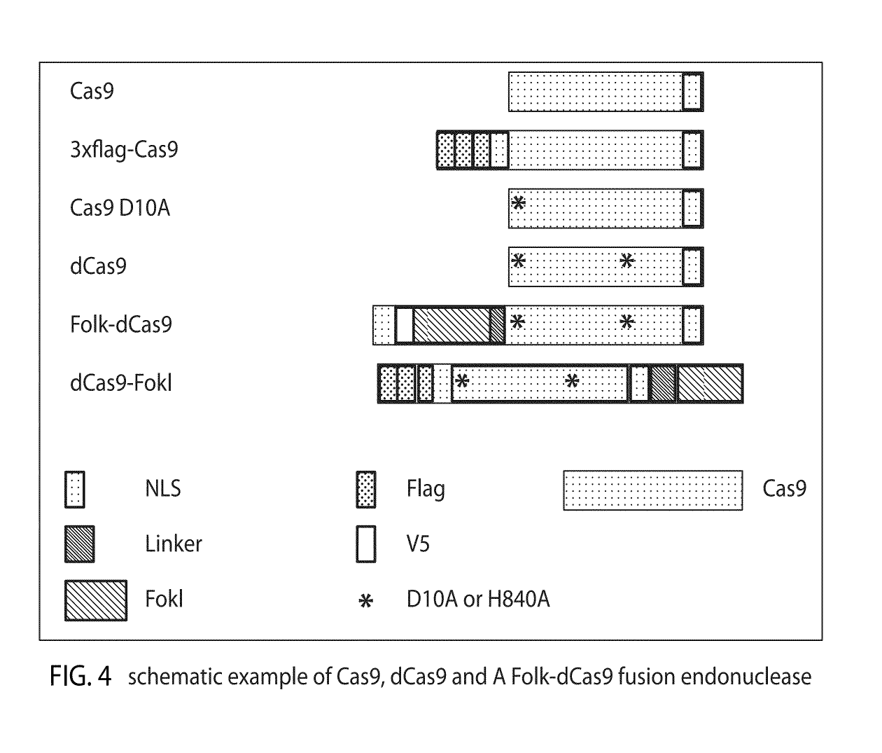

First, the DNA fragment encoding the wild type Streptococcus pyogenes Cas9 protein with a NLS at the C-terminus (SEQ ID NO: 31) was generated based on published codon optimized Cas9 sequence (Mali P, et al, Science. 2013 Feb. 15; 339 (6121):823-6) by assembling synthetic DNA fragments (gBlocks from IDT Integrated DNA Technologies) using standard PCR, restriction enzyme digestion and ligation methods. The DNA fragment was cloned into either pcDNA3.1 plasmids (Lifetechnologies) or a mouse Rosa ZFN plasmid, pVAX-ZFN73 (SAGE Labs) at the KpnI and XbaI sites to obtain pcDNA3.1/Cas9 and pVAX/3xFlag-Cas9 plasmids (FIG. 4). Both of these plasmids contain CMV and T7 promoters upstream of the Cas9 coding DNA and a polyadenylation signal sequence downstream of the Cas9 coding DNA. The CMV promoter drives Cas9 expression in mammalian cells, whereas the T7 promoter is used for in vitro RNA transcription. The resulting pcDNA3.1/Cas9 includes a NLS at the C-terminus, whereas the pVAX/Cas9 plasmid includes 3xFlag-NLS encoding sequence upstream of the Cas9 DNA in addition to the C-terminal NLS (FIG. 4). The protein sequence of a wild type Cas9 with an NLS at its C-terminus is provided in the SEQ ID NO: 31.

Secondly, a catalytically inactive Cas9 (dCas9) was created by mutating the coding sequence of the RuvC and HNH nuclease active sites of the Cas9 protein. Specifically, the above described two Cas9 plasmids underwent point mutations via substitutions of amino acid residue Asp10 to Ala (D10A), and His840 to Ala (H840A) in the Cas9 nuclease domains using standard site-directed mutagenesis methods to obtain the catalytically inactive Cas9 encoding plasmid (FIG. 4). The protein of a dCas9 without NLS sequence is provided in the SEQ ID NO: 1. A mutant Cas9 D10A, a Cas9 nickase that was only mutated at D10 site, was also generated by the same method (FIG. 4).

Next, A DNA construct encoding an NLS-V5-FokI-Linker-dCas9-NLS fusion protein, also named FokI-dCas9 in most parts of this disclosure was generated by subcloning synthetic DNA fragments (gBlocks from IDT Integrated DNA Technologies) encoding the NLS-V5-FokI-Linker into the above described pcDNA3.1/dCas9 plasmid using standard molecular cloning methods (FIG. 4). The NLS is a nuclear localization signal sequence, an example of NLS sequence is provided in SEQ ID NO: 6. The V5 is a tag that can be used for detecting the fusion protein with anti-V5 antibody. Its amino acid sequence is: GKPIPNPLLGLDST. It should be understood that V5 tag is not necessary for the function of FokI-dCas9 system.

The FokI DNA cleavage domain was placed at the N-terminus of the dCas9-NLS protein, whereas the NLS-V5 was placed at the N-terminus of FokI-Linker-dCas9-NLS coding sequence (FIG. 4). The FokI DNA cleavage domain in the FokI-dCas9 fusion protein was a modified FokI Sharkey domain (as reported in Guo et al., J. Mol. Biol. 2010; 400(1): 96-107). The respective amino acid sequence of this FokI DNA cleavage domain (Sharkey) is provided in SEQ ID NO: 9. The FokI domain in the Fok-dCas9 protein can also be a wild type FokI DNA cleavage domain, its sequence is listed in SEQ ID NO: 24.

The Linker in the fusion protein is a polypeptide between FokI domain and dCas9 protein. It is critical for the FokI-dCas9 to form a dimer when guided by an sgRNA pair. An example of the FokI-dCas9 chimeric fusion protein FokI-dCas9 (L4) that has a linker L4 is provided in the SEQ ID NOS:18 and 19. Several other FokI-dCas9 variants that only differ in Linker sequence were also created by subcloning synthetic DNA fragments encoding different Linkers (Table 1) into the FokI-dCas9 (L4) plasmid (SEQ ID NOS: 20-23. Several examples of the linkers used in the FokI-dCas9 proteins are listed in Table 1. It should be understood that linkers with other amino acid sequences could also be used with the FokI-dCas9 system.

Similarly, plasmids encoding 3xFlag-NLS-dCas9-Linker-FokI (dCas9-FokI) chimeric proteins with different Linkers were also created by subcloning synthetic DNA fragments encoding linker-FokI domain into the pVAX/3xFlag-dCas9 plasmid using standard molecular cloning methods (FIG. 4). In this type of dCas9-FokI fusion proteins, the FokI was engineered at the C-terminus of dCas9 protein (FIG. 4). These linker sequences are provided in Table 1 (SEQ ID NOS: 4-5). The sequence of a dCas9-FokI fusion protein is provided in SEQ ID NO: 2. These dCas9-FokI fusion proteins were used as controls to the FokI-dCas9 fusion proteins.

TABLE 1
FokI-dCas9, dCas9-FokI and their
linker information

| Fusion Protein | Linker | Linker Amino Acid |
|---|---|---|
| Type | Name | Sequence |
| FokI-dCas9 | L4 | GVPA |
| FokI-dCas9 | L5 | GGVPA |
| FokI-dCas9 | L8 | AGGAGVPA |
| FokI-dCas9 | L18 | AGPRGSGNGSSHGAGVPA |
| FokI-dCas9 | L28 | AGPRGSGNQGGSAASTGSGSSHGAGVPA |
| FokI-dCas9 | L40 | AGPRGSGNQGGSAASTGRGGSL |
| | | AQRSATGSGSSHGAGVPA |
| dCas9-FokI | CL42 | RTGGGSSGTGQGGSAASRGGSL |
| | | AQDVASTGGGSSGGGPRAGS |
| dCas9-FokI | CL22 | RTGGGSSGTGGGSSGGGPRAGS |

### Example 2

### FokI-dCas9 System-Mediated Genome Mutations in Mouse Rosa26 Locus

In this example, the applications of a FokI-dCas9 fusion protein to induce genome mutations in cultured mouse cells are described.

Rosa26 has been widely used as a model for inserting foreign DNA. This example uses a partial mouse Rosa26 sequence (Chr6: 113,075,754-113,076,639) (SEQ ID NO: 37) to demonstrate how the FokI-dCas9 system induces DSBs in a gene and creates mutations by the error-prone nonhomologous end joining (NHEJ) mechanism. This example also demonstrates how the spacer lengths between two sgRNA target sites and the orientation of a paired sgRNA affect the fusion protein mediated mutations.

Partial mouse Rosa26 genomic DNA sequence (886 bp) was selected from the C57BL/6 mouse genome (Chr 6:113,075,754-113,076,639) for testing FokI-dCas9 fusion protein-mediated gene editing. Specifically, the following steps were performed: (1) Engineering a FokI-dCas9 and a dCas9-FokI fusion proteins as described in example 1. The FokI-dCas9 fusion protein used in this test has a L8 linker, named FokI-dCas9 (L8). Its sequence is provided in SEQ ID NO:20. The dCas9-FokI protein has a CL42aa linker (SEQ ID NO: 2). (2) Design and synthesis of mouse Rosa26 sgRNAs. sgRNA target sites in mouse Rosa26 locus were selected for by identifying PAM (NGG, N denotes for any nucleotides) sites and using a 18-20 nt protospacer sequence upstream of the PAM site to blast the mouse genome, or by using online sgRNA design tools, such as MIT's CRISPR design tool (available at crispr.mit.edu) to choose appropriate sgRNA target sites. Protospacer sequences with the least number of matches to other sequences in the mouse genome were selected for sgRNA design. Eleven mouse Rosa26 sgRNAs were designed and used in the test and their target sites are listed in Table 2.

TABLE 2
Mouse Rosa26 sgRNA target sites

| sgRNA | | | |
|---|---|---|---|
| ID | Protospacer Sequence | PAM | Strand |
| 4 | CGCCCATCTTCTAGAAAGAC | TGG | − |
| 7 | GGCTCAGCACGCCCCTCTTG | AGG | − |
| 8 | GCAGTAGGGCTGAGCGGCTG | CGG | + |
| 9 | CCTCTTGAGGCAACTCAAGT | CGG | − |
| 11 | GGCAGGCTTAAAGGCTAACC | TGG | + |
| 13 | GGGAGTTCTCTGCTGCCTCC | TGG | + |
| 14 | GGATTCTCCCAGGCCCAGGG | CGG | − |
| 15 | TGGGCGGGAGTCTTCTGGGC | AGG | + |
| 16 | AGTCTTCTGGGCAGGCTTAA | AGG | + |
| 17 | GACTGGAGTTGCAGATCACG | AGG | − |
| 18 | GTTGCAGATCACGAGGGAAG | AGG | − |

For each sgRNA, a specific 60 nt DNA oligo comprising of a 20 nt T7 promoter at the 5′, 18-20 nt protospacer sequence downstream of the T7 promoter, and 20 nt common sequence at the 3′ (5′-GTTTTAGAGCTAGAAATAGC-3′) was synthesized and purchased from IDT Integrated DNA Technologies. An example of a 60 nt DNA oligo, the oligo for making mouse Rosa sgRNA16, is listed below, where the underlined 20 nt sequence is the T7 promoter site and the 20 nt sequence in uppercase is the protospacer sequence for sgRNA16 (5′-3′):

taatacgactcactatagggAGTCTTCTGGGCAGGCTTAAgttttagag
ctagaaatagc

An 82 nt common DNA oligo, which encodes the common sgRNA scaffold sequence (SEQ ID NO:3), was synthesized and purchased from IDT Integrated DNA Technologies. The 82 nt oligo has a 20 nt overlapping sequence with each sgRNA's 60 nt DNA oligo templates. The sequence of the 82 nt common DNA oligo is listed below (5′-3′):

AAAAAAGCACCGACTCGGTGCCACTTTTTCAAGTTGATAACGGACTAGC
CTTATTTTAACTTGCTATTTCTAGCTCTAAAAC

Next, the 82 nt common DNA oligo is annealed with an sgRNA-specific 60 nt DNA oligo to amplify the sgRNA coding DNA template via overlapping PCR using T7 primer (5′-TAATACGACTCACTATAGGG-3′) and a reverse primer (5′-AAAAAAGCACCGACTCGGTGCC-3′). The resulting 120-122 bp DNA template was purified from the PCR product. About 2 μg DNA template for each sgRNA was used for in vitro RNA transcription, using a T7 promoter-based T7 RNA polymerase in vitro transcription kit from New England Biolabs.

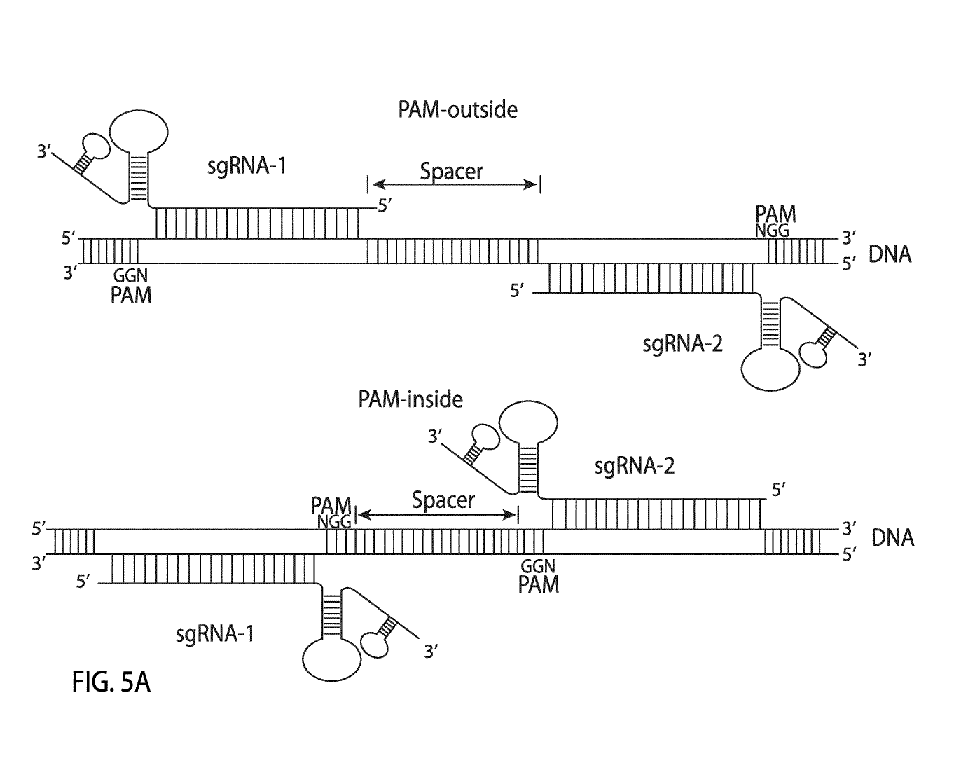

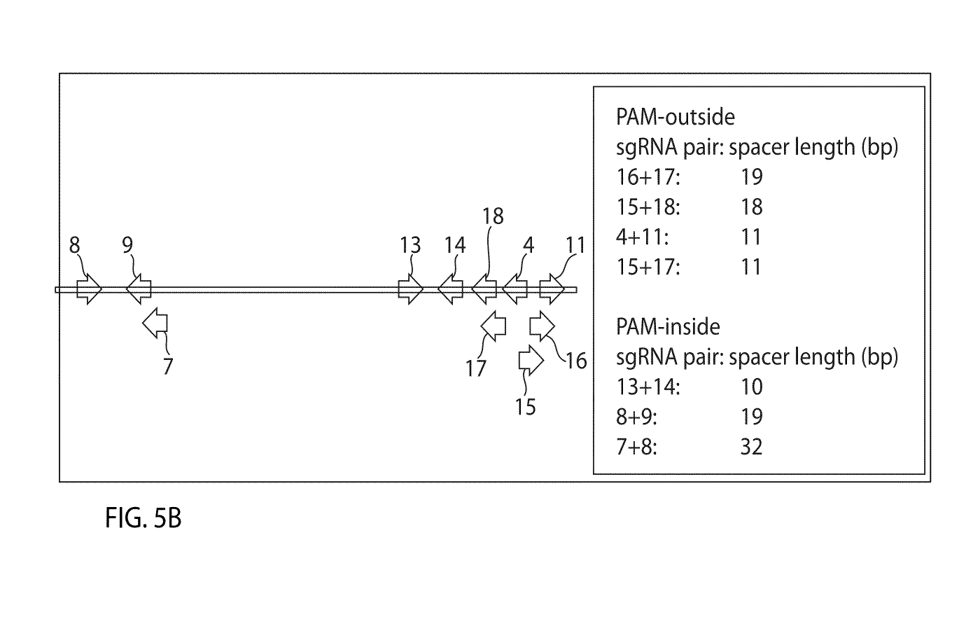

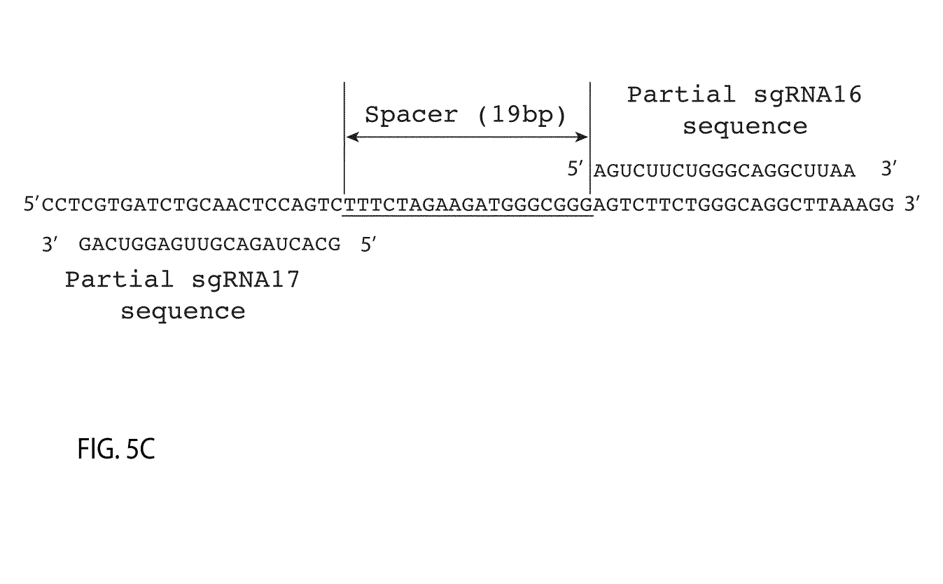

Two examples of mouse Rosa26 sgRNAs are provided below. The underlined sequence matches the Rosa26 target sequence and the lowercase sequence is a common scaffold RNA sequence (SEQ ID NO:3). sgRNA16 pairs with sgRNA17 (FIGS. 5B and C).

sgRNA16 (102 nt):
(SEQ ID NO: 32)
AGUCUUCUGGGCAGGCUUAAguuuuagagcuagaaauagcaaguu
aaaauaaggcuaguccguuaucaacuugaaaaaguggcaccgagu
cggugcuuuuuu
sgRNA17 (102 nt):
(SEQ ID NO: 33)
GACUGGAGUUGCAGAUCACGguuuuagagcuagaaauagcaaguu
aaaauaaggcuaguccguuaucaacuugaaaaaguggcaccgagu
cggugcuuuuuu

As illustrated in FIG. 5A, paired sgRNAs target different DNA strands in two different orientations, either PAM-outside or PAM-inside. Shown in FIG. 5A upper panel is a PAM-outside orientation, where the two PAMs are located outside of the two sgRNA target sites, whereas the PAM-inside orientation is illustrated in FIG. 5A, lower panel. Also illustrated in FIG. 5A is the spacer (gap) of a paired sgRNA. The Spacer is the DNA sequence between two sgRNA target sites (PAM-outside, upper panel), or between the two PAM sties (PAM-inside orientation, lower panel). The 11 mouse Rosa sgRNA target sites and their orientations are provided in FIG. 5 B. Among these 11 sgRNAs, 4 PAM-outside sgRNA pairs and 3 PAM-inside sgRNA pairs with a spacer length ranging from 10 nt to 30 nt were selected for testing for FokI-dCas9 fusion protein induced Rosa26 genomic DNA mutations. Spacer length of each sgRNA pair is listed in FIG. 5B.

An example of a paired sgRNA target site in mouse Rosa26 locus is provided in FIG. 5 C. The DNA sequence listed in FIG. 5C is a partial mouse Rosa26 locus sequence (chr6:113075997-113076061). The two PAM sites in this sgRNA pair are outside of the two sgRNA target sites. The spacer length in this sgRNA pair is 19 bp.

Next, the plasmid DNAs encoding either FokI-dCas9 (L8) or dCas9-FokI, and sgRNAs were transfected into Neuro2a cells. Specifically, Neuro2a cells cultured in Dulbecco's Modified Eagle Medium (DMEM from Hyclone) supplemented with 10% FBS, 2 mM Glutamine, and 100 U/ml penicillin/streptomycin were seeded in 24-well plates at the density of 100,000 cells per well, and incubated at 37° C. with 5% CO₂ for 18-20 h prior to transfection. Sequential transfections were employed to deliver DNA constructs encoding Cas9 or its derived fusion proteins and sgRNAs into the cells. Briefly, DNA plasmid encoding wild type Cas9, FokI-dCas9 (with L8 linker), or dCas9-FokI (with CL42 linker) were transfected into Neuro2a cells in a 24-well plate using Lipofectamine 2000 (Lifetechnologies) according to manufacturer's protocol. For each well of the 24-well plate, 1.0 μg of plasmid DNA was transfected. The transfected cells were incubated at 37° C. with 5% CO₂ in the same growth medium. Twenty-four hours post the initial transfection, either 0.75 μg single sgRNA or 1.5 μg total paired sgRNAs (each sgRNA at 0.75 μg) were transfected into the plasmid transfected cells. A negative control (Ctr) was established by transfection of Cas9 alone. The transfected cells were incubated at 37° C. with 5% CO₂ in the same growth medium before harvesting.

Genomic DNA was extracted from the transfected cells 24 h post sgRNA transfection using QuickExtract DNA extraction kit (Epicentre). Cells from each well were collected and incubated in 80 μl QuickExtract buffer at 65° C. for 10 min, 55° C. for 30 min, and 98° C. for 3 min before holding at 4° C. PCR amplification of a 457 bp fragment flanking the target sites of sgRNAs 4, 11, 13, 14, 15, 16, 17 and 18 was performed using primers Cel1F1 (5′-aagggagctgcagtggagta-3′) and Cel1R1(5′-taaaactcgggtgagcatgt-3′). Similarly, a 576 bp DNA fragment flanking the target sites of sgRNAs 7, 8 and 9 was PCR amplified using primers Cel1F2 (5′-ctgggggagtcgttttaccc-3′) and Cel1R2 (5′-agagggggaagggattctcc-3′).

Surveyor Cel-1 assay was performed to detect genome modifications. Mutations induced by Cas9 or FokI-dCas9 fusion protein at the sgRNA target site will be detected by the Cel-1 assay. Briefly, 20 μl of the PCR products flanking sgRNA target sites were denatured and reannealed to form heteroduplexes, and then incubated with 1 μl Cel-1 nuclease (Transgenomics) at 42° C. for 30 min. Cel-1 endonuclease cleaves mismatch sites in the DNA heteroduplex.

(6) The Cel-1 endonuclease treated DNA products were analyzed using a 10% PAGE-TBE gel (BioRad), stained with SYBRsafe, destained and imaged with BioRad's gel imaging system.

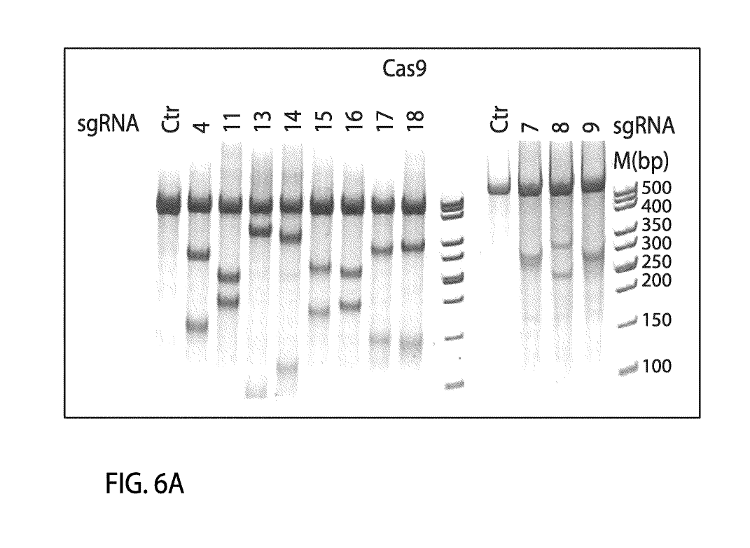

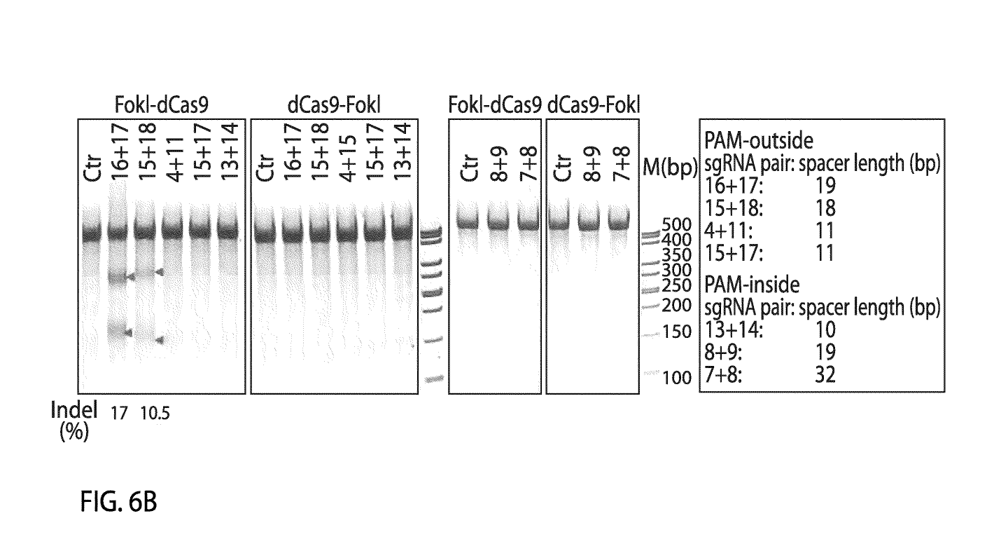

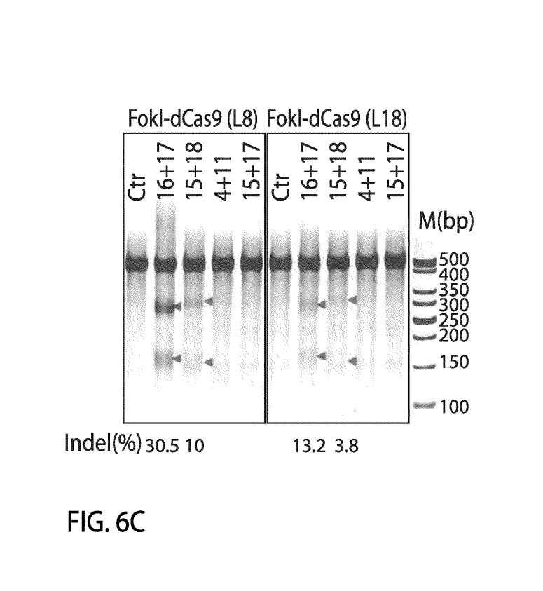

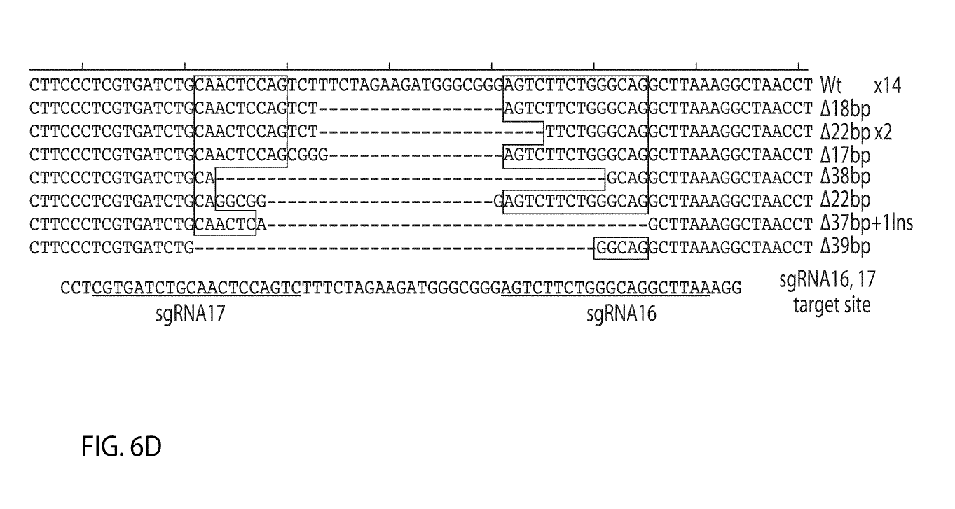

As shown in FIG. 6 A, all Cas9 and sgRNA co-transfected cells have cleaved DNA bands at the expected sizes, suggesting that these sgRNAs directed Cas9 protein to their target sites and Cas9 introduced mutations through the NHEJ pathway. As expected, the control sample (Ctr) transfected with Cas9 alone did not show any cleaved DNA bands, indicating the specificity of the assay.

It was expected that two sgRNAs in a pair, targeting two adjacent sites on the Rosa26 gene could bring the two FokI-dCas9 fusion proteins together, and if the two FokI monomers are in the appropriate orientation and distance, they could form a FokI dimer, reconstituting the FokI endonuclease activity, and leading to double-strand breaks (DSBs) in the target DNA via the NHEJ pathway.

Surveyor Cel-1 assay results showed that cleaved DNA bands were detected in samples transfected with FokI-dCas9 and sgRNA pair 16,17 (FIG. 6B). More importantly, the two band sizes match the expected 181 and 276 bp sizes. Additionally, although to a lesser extent, cleaved DNA bands were also observed in FokI-dCas9 and sgRNA pair 15,18 transfected cells at the expected 174 and 283 bp sizes. In contrast, no cleaved DNA bands were detected in other sgRNAs and FokI-dCas9 co-transfected cells, indicating that the FokI-dCas9 only induced Rosa26 mutations in cells co-transfected with sgRNA pair 16,17 or pair 15,18 (FIG. 6B).

The spacer lengths for sgRNA pairs 16,17; 15,18; 4,11 and 15,17 are 19, 18, 11 and 11 bp, respectively. All 4 pairs are in a PAM-outside orientation. The fact that there were no mutations detected in pairs 4,11 and 15,17 transfected cells suggests that spacer length in paired sgRNA target sites is critical for FokI-dCas9 mediated DNA mutation, and that a 11 bp spacer may not be enough for Fok-dCas9 dimer formation under the test conditions. Note that the cleaved DNA bands in FokI-dCas9 and sgRNA pair 16,17 or sgRNA pair 15,18 treated samples are broader than those observed in wild type Cas9 transfected samples, indicating that FokI introduces larger and more heterogeneous mutations (indels) than Cas9 does.

FIG. 6 B also demonstrated that PAM orientation is essential for FokI-dCas9 mediated DNA cleavage. As shown in FIG. 5 B, sgRNA pair 8,9 is in a PAM-inside orientation, and although the spacer length of sgRNA pair 8,9 is also 19 bp as in pair 16,17, there was no detectable mutation in pair 8,9 transfected cells, most likely due to the PAM-inside orientation (FIG. 6B). Actually, there were no mutations detected in any gRNA pairs with a PAM-inside orientation, suggesting that FokI-dCas9 activity requires the PAM-outside orientation (FIG. 6 B).

Although sgRNAs 15, 16, 17 and 18 showed efficient activity in wild type Cas9, FokI-dCas9 mediated DNA mutation frequency in pair 16,17 is much higher than that of pair 15,18, suggesting that FokI-dCas9 mediated DNA cleavage is more stringent than wild type Cas9. Even 1 bp difference in spacer length significantly affects mutation frequency. These results suggest that the spacer length and PAM orientation are important factors for FokI-dCas9 to form dimers and reconstitute FokI DNA cleavage activity.

As shown in FIG. 6 B, none of dCas9-FokI transfected cells showed detectable mutations in the Surveyor Cel-1 assay, which suggests that the FokI domain fused to the C-terminus of dCas9 protein is not able to easily form dimers.

To compare the effect of the linker length on the efficiency of FokI-dCas9 mediated mutation. Two FokI-dCas9 variants, one with Linker L8 and the other with Linker L18 were test for the efficiency of mutations. As shown in FIG. 6C, while both Fok-dCas9 variants were able to induce mutations when guided by dgRNA pairs 16, 17 and 15, 18, FokI-dCas9 (L8) is more efficient than the FokI-dCas9 (L18) suggesting that shorter linker is more efficient for these two sgRNA pairs.

To further verify the mutations induced by FokI-dCas9 fusion, the PCR products flanking the target site from a FokI-dCas9 (L18) and sgRNA16,17 co-transfected Neuro2a cells (the same as in FIG. 6 C) were TA cloned into TOPO-TA vector (Lifetechnologies), and plasmid DNA from 24 colonies were sequenced using the PCR primers described above. Sanger sequencing data demonstrated that about 33% of the colonies (8 out of 24) contain mutations at the target site (FIG. 6D). As illustrated in FIG. 6D, eight sequences with deletion mutations were observed. All mutations were at the sgRNA16,17 target site. Interestingly, all mutations are deletion mutations, with deletion sizes ranging from 17 bp to 39 bp. One mutation contains a 37 bp deletion and 1 bp insertion. These sequencing results confirm that FokI-dCas9 system generated efficient Rosa26 gene mutations when guided by sgRNA pair 16,17.

In summary, this example demonstrates that the FokI-dCas9 fusion protein is able to mediate mouse genomic DNA cleavage and induce DNA mutations at the targeting site when the paired sgRNAs are in a PAM-outside orientation with an 18 or 19 bp spacer. It also demonstrated that in the FokI-dCas9 fusion protein, the FokI domain needs to be fused to the N-terminus of dCas9 domain to mediate sgRNA-guided genome modification.

### Example 3

### FokI-dCas9 System-Mediated Human Genome Modification

In this example, FokI-dCas9 fusion protein-mediated genome mutations in human EMX1 locus in cultured human cells is described.

Specifically, a partial sequence (Chr 2: 73160831-73161367; SEQ ID NO: 38) of human gene EMX1 was selected for testing paired sgRNA guided FokI-dCas9 activity in HEK293 cells. Thirteen sgRNAs targeting human EMX1 gene were designed and made using the method described in Example 2. Among these EMX1 sgRNAs, the target sequences of sgRNAs 1, 9, 20 and 22 were based on previous publications (Ran F A, et al. Cell. 2013 Sep. 12; 154(6):1380-9), and sgRNA15S and 17S were modified from the same paper by using an 18 bp target sequence. These sgRNA target sites are listed in Table 3.

TABLE 3
Human EMX1 sgRNA target sites

| sgRNA ID | Protospacer Sequence | PAM | Strand |
|---|---|---|---|
| 4 | CGCCCATCTTCTAGAAAGAC | TGG | − |
| 7 | GGCTCAGCACGCCCCTCTTG | AGG | − |
| 8 | GCAGTAGGGCTGAGCGGCTG | CGG | + |
| 9 | CCTCTTGAGGCAACTCAAGT | CGG | − |
| 11 | GGCAGGCTTAAAGGCTAACC | TGG | + |
| 13 | GGGAGTTCTCTGCTGCCTCC | TGG | + |
| 14 | GGATTCTCCCAGGCCCAGGG | CGG | − |
| 15 | TGGGCGGGAGTCTTCTGGGC | AGG | + |
| 16 | AGTCTTCTGGGCAGGCTTAA | AGG | + |
| 17 | GACTGGAGTTGCAGATCACG | AGG | − |
| 18 | GTTGCAGATCACGAGGGAAG | AGG | − |

All EMX1 sgRNAs used in this example were in vitro transcribed from DNA templates using the same method as described in Example 2. Three FokI-dCas9 variants, namely FokI-dCas9 (L4), FokI-dCas9 (L18), FokI-dCas9 (L40), were used in this example. All 3 FokI-dCas9 constructs were engineered and prepared as described in Example 1. The only difference among these 3 variants are their linkers. The sequences of these linkers are provided in Table 1.

Similar steps as described in Example 2 were performed to test these FokI-dCas9 variant-mediated EMX1 mutations. Briefly, HEK293 cells maintained in DMEM growth medium with 10% FBS, and 2 mM L-glutamine and 1 mM sodium pyruvate were seeded in 24-well plates at the density of 120,000 cells per well 18-20 h prior to transfection. First, 0.6 μg Cas9 or FokI-dCas9 DNA plasmid per well of a 24-well plate was transfected in the HEK293 cells using Lipofectamine 2000. The next day, either 0.65 μg of single EMX1 sgRNA or 1.3 μg total of paired EMX1 sgRNAs (0.65 μg for each sgRNA) were transfected using Lipofectamine 2000. The transiently transfected cells were harvested 24 h post sgRNA transfection, and genomic DNA from each well of the 24-well plate was extracted using the method as described in Example 2. PCR amplification of a 537 bp fragment flanking the target sites of the 13 EMX1 sgRNAs was performed using primers EMX Cel1F1 (5′-cagctcagcctgagtgttga3′) and EMX Cel1R1 (5′-agggagattggagacacgga-3′). Surveyor Cel-1 assay was employed to detect mutations induced by FokI-dCas9 fusion proteins.

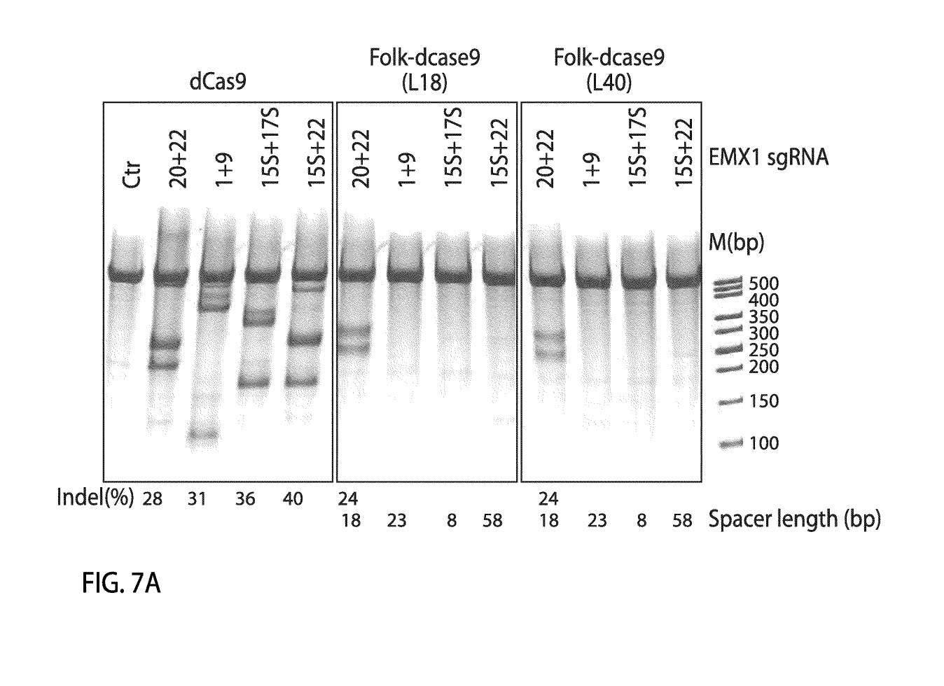

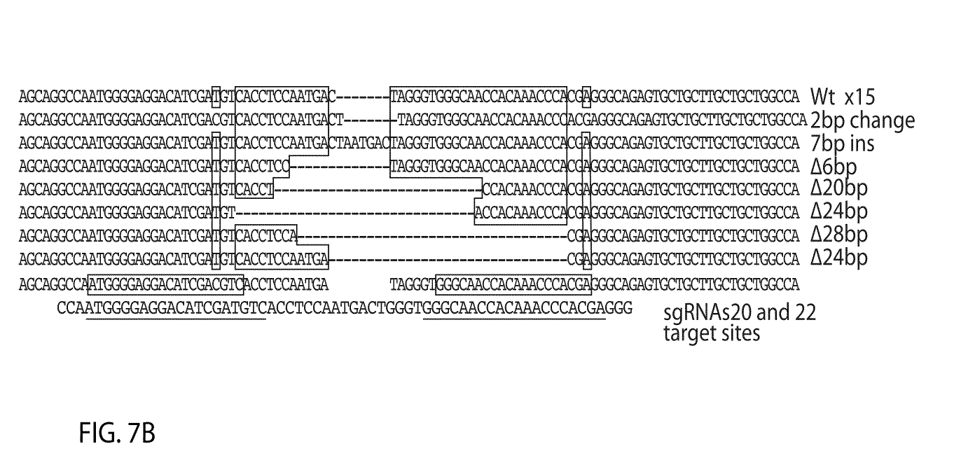

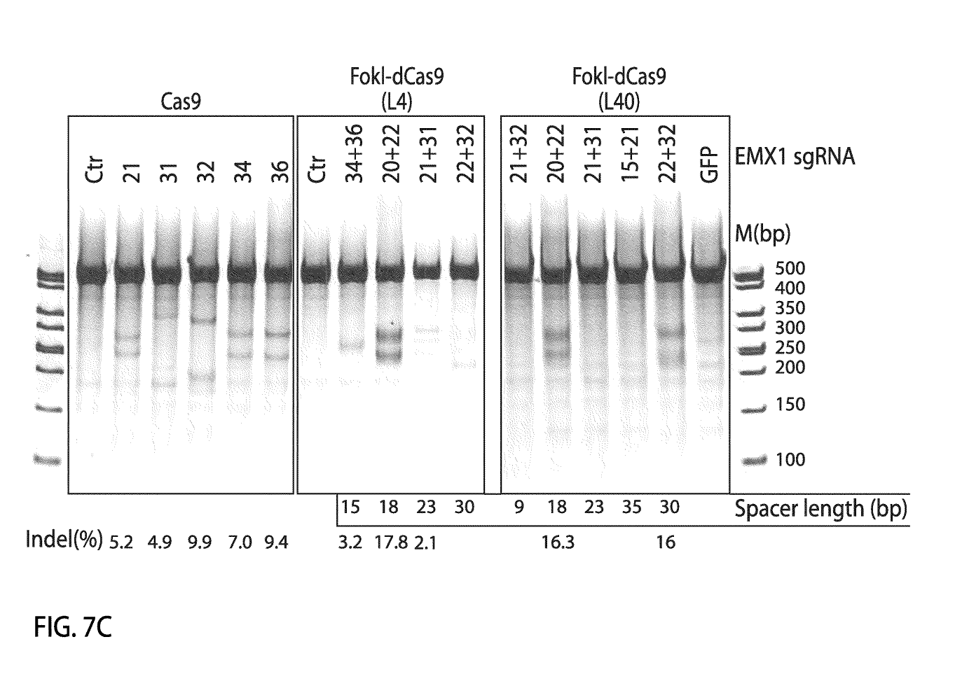

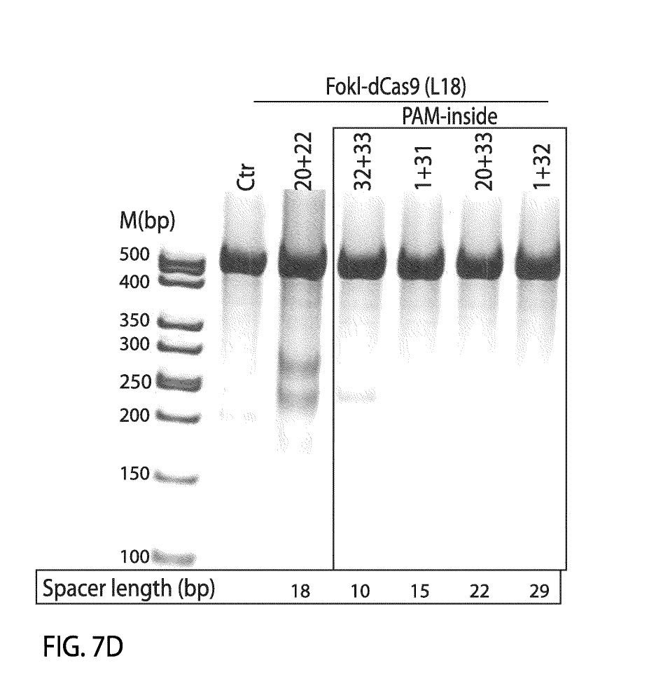

As illustrated in FIG. 7A, four EMX1 sgRNA pairs and 2 FokI-dCas9 variants, L18 and L40, were tested in this experiment first. These 4 EMX1 sgRNA pairs are all in PAM-outside orientation and with spacer lengths of 8, 18, 23 and 58 bp as indicated in the picture. As expected, cleaved DNA bands were detected in all wild type Cas9 and sgRNA co-transfected samples at the expected sizes, indicating that all of those sgRNAs were able to guide Cas9 protein to their target (FIG. 7A, left 5 lanes). Importantly, two cleaved DNA bands were detected in samples co-transfected with either L18 or L40 FokI-dCas9 and EMX1 sgRNA pair 20,22, at the expected 290 and 247 bp band sizes. These results are consistent with the results obtained from Example 2, further confirming that these two FokI-dCas9 variants were able to mediate human EMX1 gene mutations in HEK293 cells when guided by sgRNA pairs with 18 bp spacer length and in PAM-outside orientation. Not surprisingly, no noticeable cleaved DNA bands were detected in samples transfected with other EMX1 sgRNA pairs, suggesting that under the testing conditions, the spacer lengths of 8, 23, and 58 bp are not suitable for mediating FokI-dCas9 dimerization at the target site. These results also confirm that FoKI-dCas9 mediated gene targeting is more stringent.

To verify FokI-dCas9 mediated mutations in the EMX1 site, a TA-cloning approach was employed to clone the 537 bp PCR amplicons flanking the EMX1 sgRNA target site into Topo TA cloning vector (Lifetechnologies). PCR amplicons from FokI-dCas9 (L18) and sgRNAs 20 and 22 co-transfected samples were selected for TA-cloning. Plasmid DNAs from 24 colonies were sequenced by Sanger sequencing using PCR primer EMX Cel1F1 and EMX Cel1R1, respectively. Sequencing results demonstrated that there were 7 different mutations in the total of 22 readable EMX1 sequences. As illustrated in FIG. 7 B, all 7 mutations are located in the sgRNA 20 and 22 target site. Most of these mutations are deletion mutations, ranging from 6 bp to 28 bp deletions, with only one 7 bp insertion mutation (FIG. 7 B). These results confirm that FokI-dCas9 fusion protein guided by sgRNA 20 and 22 mediated EMX1 mutations at the target site.

To test whether different FokI-dCas9 variants with different linkers may be suitable for different spacer lengths, additional EMX1 sgRNA pairs with different spacer lengths were co-transfected with FokI-dCas9 (L4 or L40) into HEK293 cells. These EMX1 sgRNA pairs are all in PAM-outside orientation. Surveyor Cel-1 assay results showed that all of these EMX1 sgRNAs were able to guide Cas9 to induce EMX1 gene mutations at their target sites (FIG. 7 C). As expected, cleaved DNA bands were detected in the samples co-transfected with FokI-dCas9 and EMX1 sgRNA pair 20, 22 in both L4 and L40 groups. Importantly, two cleaved DNA bands were observed in the samples co-transfected with sgRNA pair 22,32 and FokI-dCas9 (L40), but not in the FokI-dCas9 (L4) variant. Furthermore, these 2 cleaved DNA bands match the expected 296 and 241 bp sizes (FIG. 7 C, left panel). These results demonstrate that sgRNA pairs with 30 bp spacer length are suitable for FokI-dCas9 with a longer linker.

Interestingly, in sgRNA pair 34,36 and FokI-dCas9 (L4) transfected cells, there was a clear, albeit weak DNA band at the size around 270 bp (FIG. 7 C). This size matches the expected cleaved DNA sizes at 268 and 269 bp for this sgRNA pair. These results demonstrate that FokI-dCas9 with linker L4 can also mediate DNA cleavage when guided by a gRNA pair with a 15 bp spacer length, although it may be less efficient under the testing conditions.

The expected cleaved DNA bands for sgRNA pair 21,31 are 313 and 224 bp. There are faint bands at the expected size in the samples from sgRNA pair 21,31 and FokI-dCas9 (L4) transfected cells (FIG. 7 C), which indicates that there might be some mutations mediated by FokI-dCas9 and sgRNA pairs with a 23 bp spacer length. However, these mutations are less frequent under the test conditions.

Results from Example 2 suggest that sgRNA pairs with PAM-inside orientation are not suitable for inducing FokI-dCas9 mediated mutations. To confirm this observation, 4 EMX1 sgRNA pairs with PAM-inside orientation were tested in HEK293 cells, along with the PAM-outside pair sgRNA 20 and 22. As illustrated in FIG. 7 D, no clear cleaved DNA bands at the expected sizes were detected in samples transfected with FokI-dCas9 (L18) and these 4 PAM-inside sgRNA pairs. The expected cleaved DNA sizes for sgRNA pair 32,33 are 339 and 198 bp, thus the faint band around 230 bp in sgRNA pair 32,33 transfected cells was not generated from a FokI-dCas9 mediated mutation. In contrast, intense cleaved DNA bands were shown in sgRNA 20,22 co-transfected sample at the expected size. These results further suggest that sgRNA pairs with PAM-inside orientation are not suitable for inducing FokI-dCas9 mediated gene targeting.

Taken together, this example demonstrates that FokI-dCas9 induces human gene mutations when guided by sgRNA pairs with spacer lengths of 15, 18 and 30 bp. It also demonstrated that FokI-dCas9 with different linkers may require sgRNA pairs with different spacer lengths.

The data from Examples 2 and 3 have demonstrated that FokI-dCas9 is able to cleave genomic DNA when guided by two sgRNAs separated by 15, 18, 19 or 30 bp apart and in a PAM-outside orientation. It should be noted that paired gRNAs with spacer lengths of 16 and 17 bp should also be able to guide FokI-dCas9 to generate genomic modifications. As the cleavage efficiency is higher with the paired sgRNA with 19 bp spacer length, it is also likely that any gRNA pairs with spacer length close to 19 bp, such as 20, 21 or even 22 bp, can also guide the FokI-dCas9 protein to induce genome modifications.

### Example 4

### FokI-dCas9 System-Mediated Genome Modifications are Highly Specific

In this example, the specificity of the FokI-dCas9 mediated gene mutations is demonstrated.

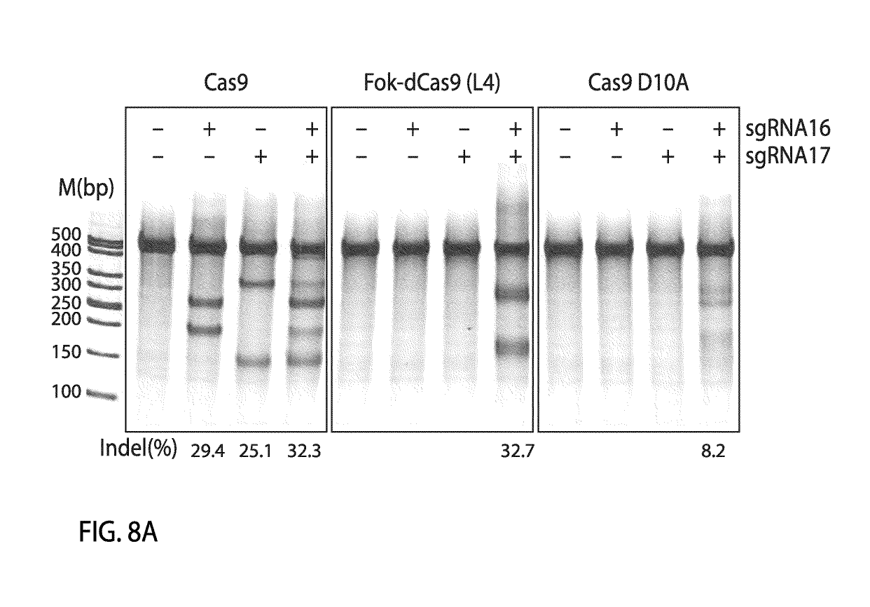

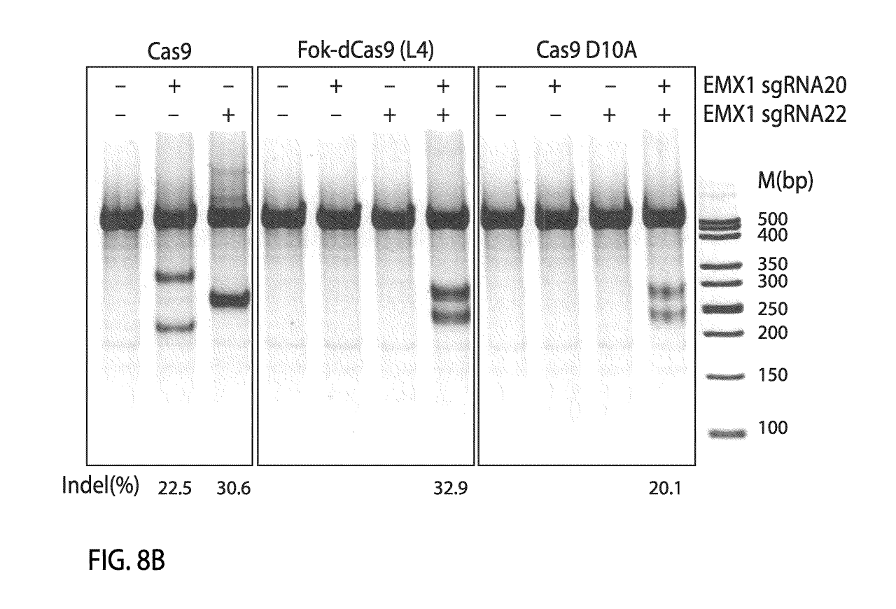

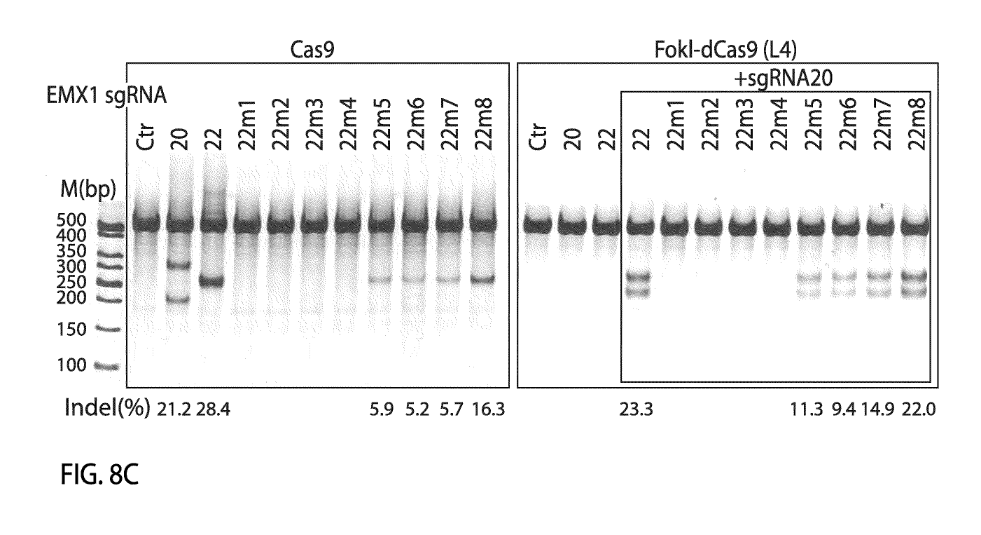

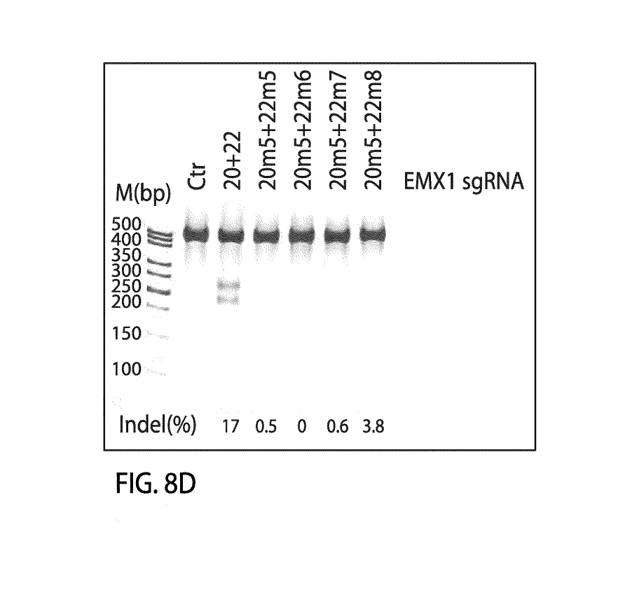

Monomeric FokI DNA cleavage domain is not able to cleavage DNA. Therefore, it is expected that FokI-dCas9 should not cleave DNA when guided by a single sgRNA, To demonstrate this hypothesis, Surveyor Cel-1 assay results from single and paired sgRNA guided FokI-dCas9 mediated gene mutation in both mouse Rosa26 and human EMX1 genes were provided. The experiment steps for this example were the same as those described in the Examples 2 and 3, but using either single or paired gRNAs to test FokI-dCas9 specificity. As illustrated in FIG. 8A, single mouse Rosa26 sgRNA 16 or 17 was able to efficiently guide Cas9 to induce Rosa26 mutations at their target sites in mouse Neuro2a cells, but no cleaved DNA bands were detected in samples from cells co-transfected with FokI-dCas9 and a single sgRNA, either sgRNA 16 or 17. The FokI-dCas9 induced mutations were only detected when both sgRNAs 16 and 17 were co-transfected (FIG. 8A). Similar results were obtained in HEK293 cells. As shown in FIG. 8 B. single EMX1 sgRNA, neither sgRNA20 nor sgRNA22 alone, was able to guide FokI-dCas9 to induce mutations, whereas highly efficient mutations were observed when both sgRNA 20 and 22 were co-transfected into the cells. These results demonstrated that FokI-dCas9 mediated genome modifications require two sgRNAs in a pair.

To further confirm the specificity of FokI-dCas9 mediated genome modification, a series of mismatch sgRNAs were designed based on human EMX1 sgRNAs 20 and 22. These mismatch sgRNAs were designed to have consecutive 2 nt mismatches to the original sgRNAs 20 and 22 protospacer sequences. Their target sequences are listed in Table 4. The sequences in lower case are mismatches compared to their on-target sgRNAs protospacer sequences.

TABLE 4
Mismatch sgRNAs for targeting EMX1 sgRNAs20
and 22 target sites

| sgRNA ID | Protospacer Sequence | PAM | Strand |
|---|---|---|---|
| 22 | GGGCAACCACAAACCCACGA | GGG | + |
| 22m1 | GGGCAACCACAAACCCACct | GGG | + |
| 22m2 | GGGCAACCACAAACCCtgGA | GGG | + |
| 22m3 | GGGCAACCACAAACggACGA | GGG | + |
| 22m4 | GGGCAACCACAAtgCCACGA | GGG | + |
| 22m5 | GGGCAACCACttACCCACGA | GGG | + |
| 22m6 | GGGCAACCtgAAACCCACGA | GGG | + |
| 22m7 | GGGCAAggACAAACCCACGA | GGG | + |
| 22m8 | GGGCttCCACAAACCCACGA | GGG | + |
| 20 | GACATCGATGTCCTCCCCAT | TGG | − |
| 20m5 | GACATCGATGagCTCCCCAT | TGG | − |
| 20m6 | GACATCGAacTCCTCCCCAT | TGG | − |
| 20m7 | GACATCctTGTCCTCCCCAT | TGG | − |
| 20m8 | GACAagGATGTCCTCCCCAT | TGG | − |

Using a similar experiment procedure as described in Example 3, EMX1 sgRNA 20 or 22, along with one of these mismtach sgRNAs, either single sgRNA, or in a pair as indicated in the FIG. 8 C, were tested for their ability to induce mutations in EMX1. Surveyor Cel-1 assay results show that matches in the first 8 nt immediately upstream of the PAM site in sgRNA protospacer sequences did not generate any mutations induced by both wild type Cas9 and FokI-d Cas9, whereas mismatches in the 9^{th} to 14^{th} nt upstream of the PAM sequence significantly reduced FokI-dCas9 induced mutation frequency, as in wild type Cas9 (FIG. 8C). Furthermore, when both sgRNAs in an sgRNA pair contain 2 nt mismatches, there were hardly any mutations detected by Surveyor Cel-1 assay even the mismatches in the 2 sgRNAs are in 9^{th} to 14^{th} nt upstream of PAM site (FIG. 8D). These results established that FokI-dCas9 mediated genome modification not only requires two sgRNAs, but also requires each sgRNA to match its target site sequence. Otherwise, the mutation frequency will be significantly affected

### Example 5

### FokI-dCas9 Facilitated Targeted Integrations

Having demonstrated the efficient and specific gene mutations induced by FokI-dCas9, the ability of FokI-dCas9 to facilitate targeted integrations is described here.

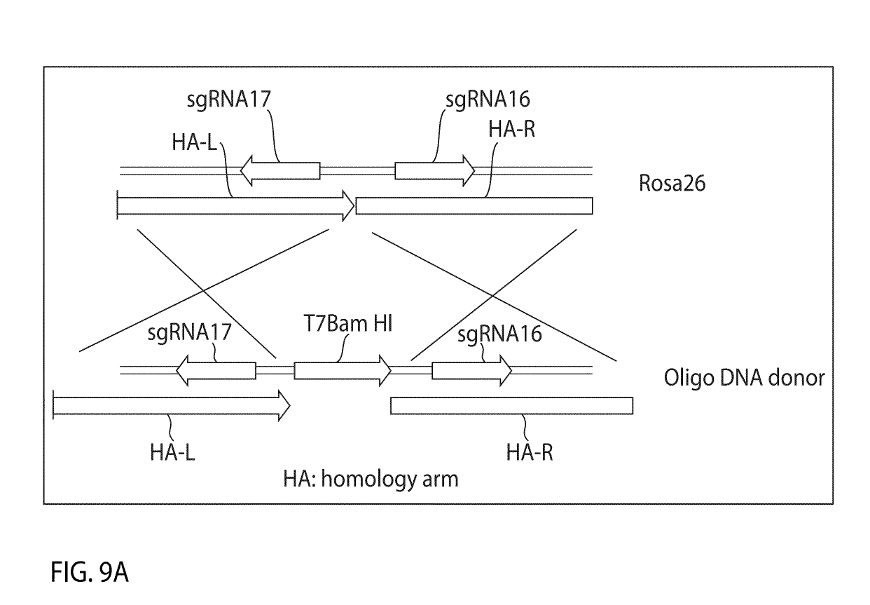

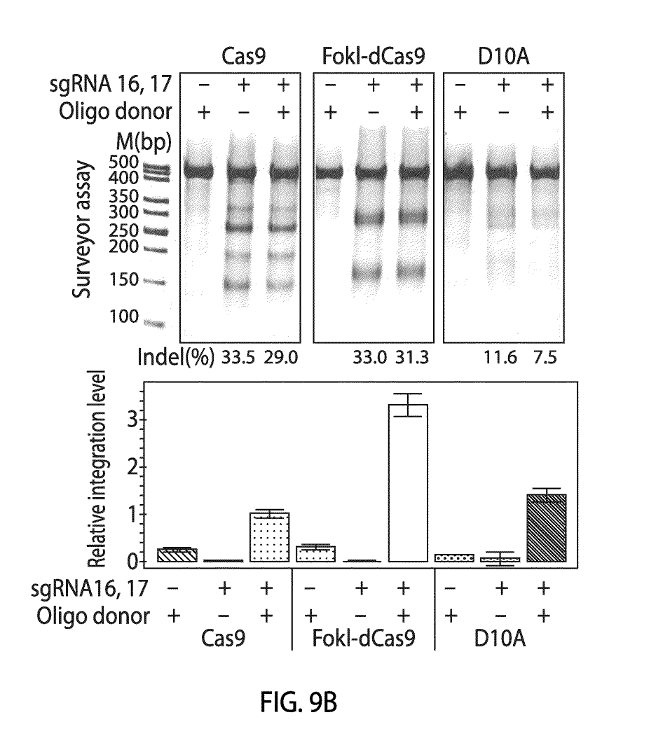

To test the efficiency of FokI-dCas9 mediated targeted DNA integration (knock in), a DNA oligo donor was designed to target mouse Rosa26 locus at sgRNAs 16 and 17 target site (FIG. 9A). This donor has 60 nt of homology arms on both sites, and a 24 nt insertion sequence that contains a BamHI site and a T7 promoter sequence, which can used for detecting targeted integration. The sequence of this olido donor is provided (SEQ ID NO: 40). This single-stranded DNA oligo was synthesized and purchased from IDT Integrated DNA Technologies.

The oligo donor DNA was co-transfected with mouse Rosa26 sgRNA pair 16, 17 as described in Example 2. Briefly, Neuro2a cells grown in 24-well plate were first transfected with 1 μg of either Cas9, FokI-dCas9, or Cas9 D10A DNA plasmid. The next day, 1.5 μg of sgRNA pair 16, 17, and 0.5 μg DNA oligo donor, either alone or in combination, was transfected into Neuro2a cells. The cells were collected 24-30 h post sgRNA transfection, and genomic DNA extract was prepared for testing mutation efficiency by Surveyor Cel-1 assay, and for targeted integration efficiency by quantitative junction PCR.

Targeted DNA integration efficiency was assayed by quantitative PCR (qPCR) using T7 primer (5′-gaataatacgactcactataggg-3′) and a reverse primer Cel-1R (5′-caaaaccgaaaatctgtggg-3′) that binds downstream of the targeted integration site. This primer pair can only amplify DNA from a targeted integration site. Reference gene primers were from further downstream of the target site. qPCR was performed using SYBRGreen Jumpstart kit (Sigma-Aldrich) according to manufacturer protocol on BioRad's plate reader.

As demonstrated in FIG. 9B, FokI-dCas9 mediated efficient DNA cleavage in Neuro2a cells. More importantly, qPCR results demonstrate that FokI-dCas9 induced targeted integration rate is 2 times higher than that of Cas9 (FIG. 9B, lower panel). Given that wild type Cas9 has been successfully used for mediating targeted integrations in diverse types of cells and animal models, the FokI-dCas9 system will be more useful to mediate targeted integrations, including point mutation, insertion, deletion, replacement and other targeted modifications in various organisms. These results demonstrated that FokI-dCas9 not only is able to efficiently mediate DNA cleavage, but is also useful in facilitating targeted integrations.

### Example 6

### Application of FokI-dCas9 System in Mouse Embryos

Having shown efficient and specific genome modifications-mediated by FokI-dCas9 in cultured cells, efficient genome modification in mouse embryos mediated by FokI-dCas9 is demonstrated in this example. The following steps were performed.

(1) FokI-dCas9 mRNA preparation. The pcDNA3.1/FokI-dCas9 (L4) plasmid was linearized downstream of its coding sequence by XbaI digestion, and 1 μg of purified linearized plasmid DNA was used for in vitro transcription using MessageMaxT7 Capped Message Transcription kit (Epicentre Biotechnologies) according to manufacture protocol. After 1.5 h, 37° C. incubation, a poly A tailing reaction was performed using A-Plus poly (A) polymerase tailing kit (Epicentre Biotechnologies) for 1 h. Then, the FokI-dCas9 mRNA was purified and dissolved in injection buffer (1 mM Tris pH7.4, 0.25 mM EDTA, 0.02 μm filtered).

(2) Pronuclear microinjection into fertilized mouse embryos. Sixty ng/μl FokI-dCas9 mRNA, and 20 ng/μl mouse Rosa26 sgRNA 16 and 17 were co-injected into pronuclei of fertilized mouse embryos according to SAGE Labs' standard protocol. The injected embryos were cultured in M2 injection medium and incubated at 37° C., 5% CO2 for 2-3 days to develop into multi-cell embryos.

(3) Surveyor Cel-1 assay was employed to genotype the injected embryos. Embryo genomic DNA was extracted in quickextraction buffer. Cel-1 PCR and Surveyor assay were performed according to the methods described in Example 2.

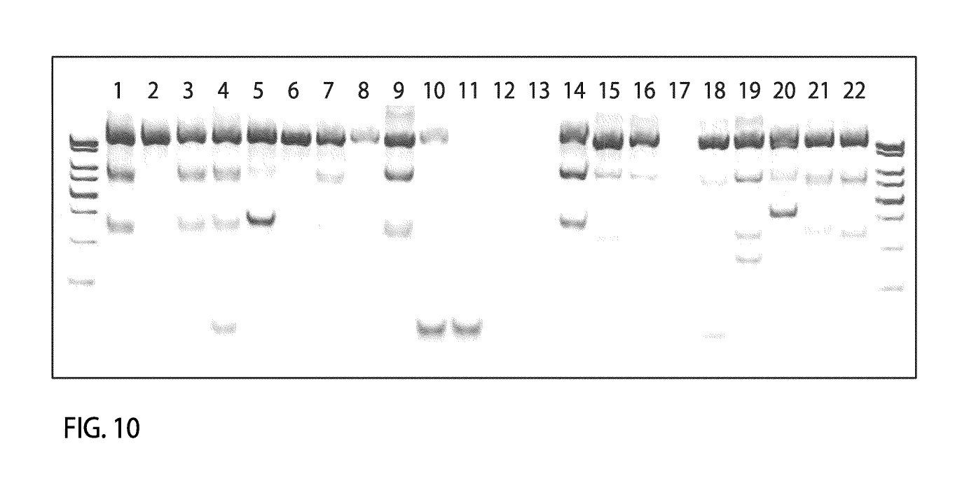

Approximately 50% of the injected mouse embryos developed into a multi-cell stage. Surveyor assay results showed that 83% embryos have cleaved DNA bands (FIG. 10), indicating that their genomes at the sgRNAs 16,17 target site underwent mutations induced by FokI-dCas9. Interestingly, the mutation frequency detected in embryos was much higher than those obtained in transiently transfected cultured cells. There are 3 samples in FIG. 10 that do not have any DNA amplicons. This could be due to biallelic large deletion that cannot be amplified by the testing primer set, or it is also possible that the genomic DNA was too dilute in those samples because these samples were from embryos that remained in the one-cell stage. Nevertheless, these embryo results demonstrate that FokI-dCas9 is able to mediate genome modification in mouse embryos at a very high efficiency.

### Example 7

### FokI-dCas9 and ZFN Hetero Dimer Mediated Genome Modifications

The above examples demonstrated efficient and specific genome modifications mediated by FokI-dCas9 fusion protein. However, the high specificity also suggests that it might not be easy to find a good sgRNA pair in a specific target region, especially when the target region is small. To overcome this issue, a FokI based heterodimer approach was introduced. An example of the FokI-dCas9 and ZFN heterodimer mediated gene modification is provided in this example.

As illustrated in FIG. 2, it was expected that a FokI-dCas9 guided by an sgRNA and a ZFN targeting the adjacent region could form a FokI heterodimer to create DSBs and mediate genome modifications. To demonstrate this model, a combination of ZFN and a single sgRNA guided FokI-dCas9 was tested in mouse Neuro2a cells. The sgRNAs used in this example were mouse Rosa sgRNAs 17, 18 that were described in Example 2. The ZFN used in the test were ZFN73Sk and ZFN77Sk, which were modified from SAGE Labs' and Sigma-Aldrich's mouse Rosa ZFN 73 and 77 bp replacing the original Hi-Fi FokI domain with the FokI Sharkey domain (SEQ ID NO: 9). The binding site of this ZNF73Sk is 5′-TGGGCGGGAGTC-3′. The sequence of the modified ZFN73Sk is listed in SEQ ID NO: 39. The ZFN73Sk construct was prepared in both plasmid and mRNA formats. The ZFN73Sk mRNA was prepared using the method described in Example 6.

In the first test, Neuro2a cells grown in a 24-well plate were co-transfected first with 0.8 μg of FokI-dCas9 plasmid and 0.6 μg of ZFN73SK plasmid using lipofectamine 2000 (Lifetechnologies). Two FokI-dCas9 variants, L8 and L18, were used in the test. The next day, either 0.75 μg of mouse Rosa sgRNA17 or 0.75 μg of sgRNA18 was transfected in the FokI-dCas9 and ZFN73Sk co-transfected cells. ZFN77Sk, which forms a dimer with ZFN73Sk, was also transfected in some wells to serve as a positive control. These transfected cells were harvested 24 h post sgRNA transfection and DNA extract was prepared using the same method as described in Example 2. Surveyor Cel-1 assay was employed.

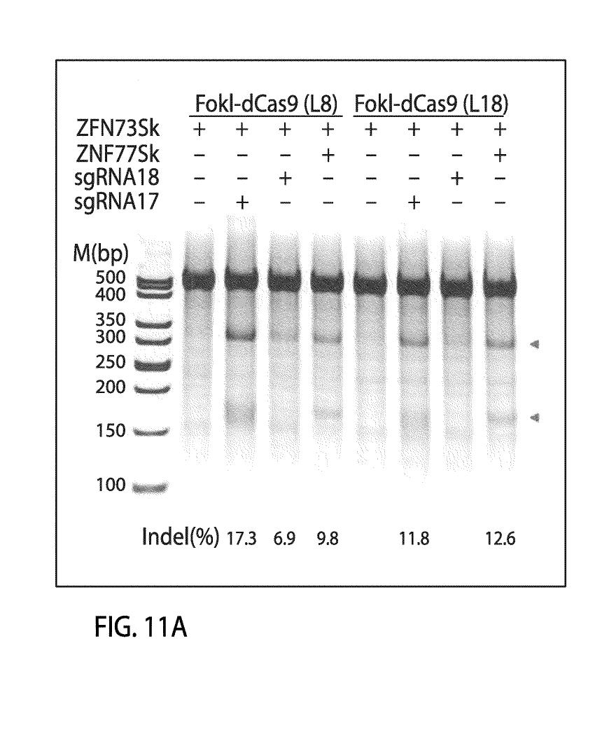

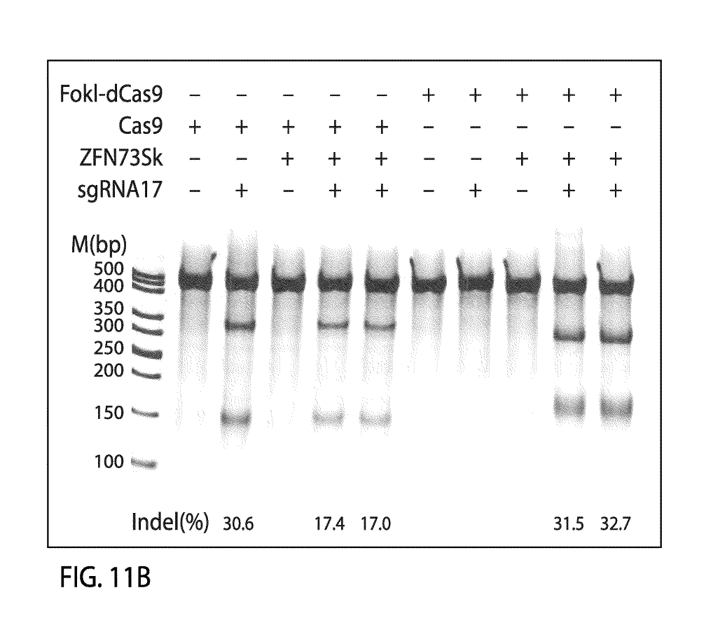

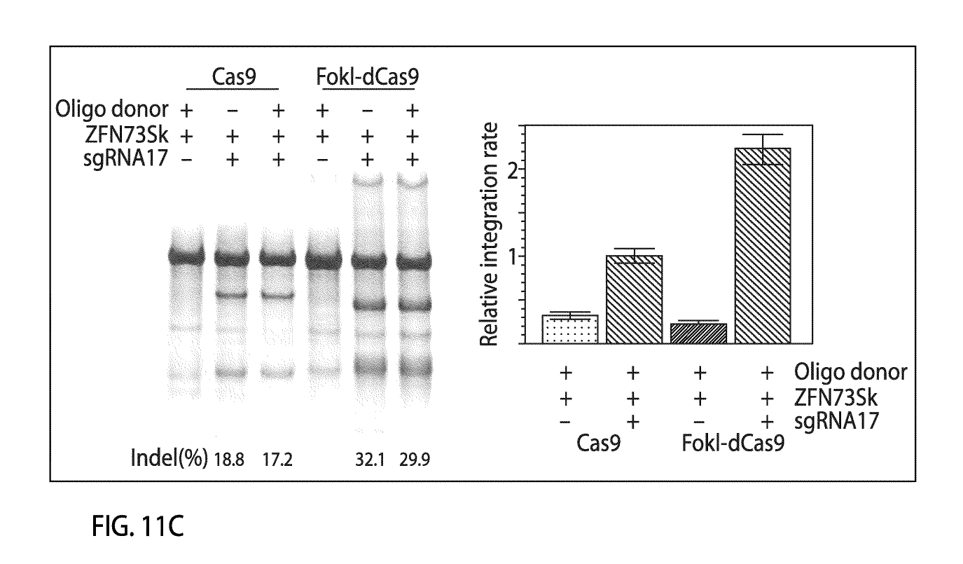

As illustrated in FIG. 11A, Surveyor assay gel demonstrated that co-transfection of ZFN73Sk and FokI-dCas9 was not able to create any mutations in the absence of sgRNA. However, two cleaved DNA bands were observed in samples from the cells co-transfected with ZFN73Sk and FokI-dCas9 plus either sgRNA17 or sgRNA18. The expected cleaved DNA band sizes are 280 and 177 bp for sgRNA17 and ZFN73Sk pair, and 283 and 174 bp for sgRNA18 and ZFN73Sk pair. Clearly, the observed DNA bands match the expected sizes. These results indicate that the FokI-dCas9 and ZFN73Sk did form a FokI dimer and cleaved the target DNA as designed. Interestingly, sgRNA17 and ZFN73Sk pair showed stronger bands than sgRNA18 and ZFN73Sk pair, possibly due to their different spacer length between the ZFN binding and sgRNA target sites. sgRNA17 and ZFN73 target sites are 11 bp apart, whereas sgRNA18 and ZFN73 target sites are 18 bp apart.

Shown in FIG. 11B are the Surveyor assay results from another test. It is similar to the first test, but with slight modifications. Briefly, Neuro2a cells were first transfected with either 1.0 μg of Cas9 or FokI-dCas9. The next day, cells were further transfected with 0.75 μg sgRNA17 or 0.75 μg ZFN73Sk mRNA, either alone or in combination, as indicated in FIG. 11B. The cells were collected 24 h post sgRNA transfection and DNA extract prepared as described in the first test. Surveyor Cel-1 assay gel demonstrated that when guided by sgRNA17, FokI-dCas9 and ZFN73Sk did form a dimer and induced mutations at the target site. Interestingly, FokI-dCas9 and ZFN73Sk mediated mutation frequency is similar to, or even slightly higher, than that of the Cas9 and sgRNA17 pair (FIG. 11B).

In the third test, the ability of FokI-dCas9 and ZFN heterodimer to facilitate targeted DNA integration is investigated. This test is similar to the second test, but a single stranded DNA oligo donor was added to test targeted integration efficiency. The oligo donor is the same one as described in Example 5 (SEQ ID NO: 40). Specifically, the Neuro2a cells grown in 24-well plates were transfected with 1.0 μg Cas9 or FokI-dCas9. On the next day, 0.75 μg sgRNA17, 0.75 μg ZFN73Sk mRNA, and 0.5 μg oligo donor DNA, were transfected, either alone or in combination, as indicated in FIG. 11C. Genomic DNA was extracted and Surveyor Cel-1 assay was performed as described. The same qPCR that was described in Example 5 was employed for the four samples with oligo donor to quantitatively amplify the targeted integration junction products.

As expected, the Surveyor assay results confirm the mutations induced by FokI-dCas9 and ZFN dimer (FIG. 11C, left panel). Since there is no junction PCR amplification in samples without donor as shown in FIG. 9B in Example 5, only the four samples with oligo donor were selected for qPCR to check for integration efficiency. As demonstrated in FIG. 11C, qPCR for targeted integration junction products demonstrated that the targeted integration rate mediated by FokI-dCas9 and ZFN dimer is more than twice as that of Cas9 and sgRNA17 mediated integration.

Taken together, results from this example demonstrate that the FokI-dCas9 and ZFN dimer is not only able to generate mutations via NHEJ, but can also facilitate targeted DNA integrations similar to how ZFNs and TALENs do. It should be noted that the 2 sgRNA worked in the test are also in PAM-outside orientation. As the PAM-inside orientation did not work in Fok-dCas9 mediated genome mutations. This PAM-outside orientation is the preferred sgRNA orientation in the Fok-dCas9/ZFN heterodimer system.

### Example 8

### FokI-dCas9 and ZFN Heterodimer Mediated Genome Modification in Mouse Embryos

In this example, the application of FokI-dCas9 and ZFN heterodimer to induce mouse gene mutations in mouse embryos is described. The experimental procedures for this test are similar to those described in Example 6, except for that instead of using two sgRNAs in a paired format, sgRNA17 and ZFN73Sk mRNA are paired.

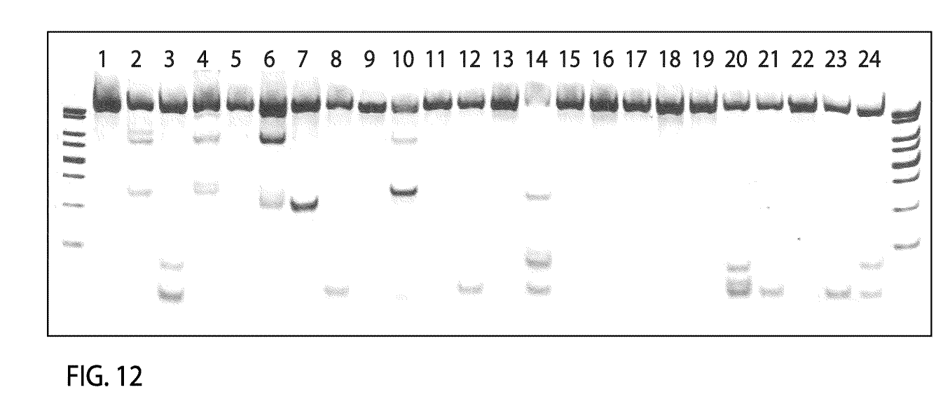

Briefly, 60 ng/μl FokI-dCas9 (L4) mRNA, 20 ng/μl mouse Rosa sgRNA17 and 20 ng/μl ZFN73Sk mRNA were co-injected into pronuclei of fertilized mouse embryos. The injected embryos were incubated for 3 days before extracting genomic DNA for genotyping. Surveyor Cel-1 assay was employed to detect the mutations in the target site. As illustrated in the FIG. 12, about 25% of the embryos have cleaved DNA bands at the expected size, indicating that those embryos have small insertion/deletion mutations at the target site. Additionally, about 30% of the embryos have smaller parental bands, which could be due to large deletion. Together, nearly half of the injected embryos have mutations. Therefore, these results demonstrated that FokI-dCas9/ZFN dimer is able to create mutations in embryos. As demonstrated in cultured cells, FokI-dCas9 and ZFN heterodimer is also suitable for generating targeted integrations in embryos when a donor DNA is provided.

Although Examples 7 and 8 were all based on the FokI-dCas9 and ZFN dimer, the concept and applications are also applicable for FokI-dCas9 and TALEN heterodimer, as both TALENs and ZFNs are based on a FokI dimerization mechanism. The FokI domain from TALENs should also be able to form a dimer with the FokI domain from FokI-dCas9 to mediate genome editing as described in the model in FIGS. 3A and B. The combination of FokI-dCas9 with ZFN and TALEN will grant scientists the ability to modify any sequence in the genome.

This heterodimer system can also be used for testing individual ZFN or TALEN. Previously, there was no easy method to test whether an individual ZFN or TALEN is active, they must be tested in a pair. As it is easy to test whether a sgRNA is active, it will be possible to use the FokI-dCas9 and ZFN or TALEN heterodimer to test individual ZFN or TALEN. This system can facilitate ZFN and TALNE designs.

In view of the above, the chimeric fusion proteins and methods described herein allow for gene targeting with higher specificity when compared to the original CRISPR/Cas9 system while maintaining the simplicity of the original CRISPR/Cas9 system. A significant advantage of the present described system over the original CRISPR/Cas9 system is that the specificity of the present system is significantly improved, because in the present system, its specificity can be directed by two different sgRNA sequences, as well as two PAM sites, whereas in the original CRISPR/Cas system, its specificity only depends on one sgRNA and one PAM site. Another advantage is that reprogramming of the present chimeric fusion protein to target different DNAs does not require re-engineering a sequence-specific DNA binding domain as the sequences of the sgRNA can be changed to target a different target DNA, which is much easier than reconstructing ZFNs or TALENs. The present system can also be paired with nucleases such as, for example, ZFNs or TALENs, to target basically any DNA of interest where DNA binding using different binding sites in the target DNA is needed.

When introducing elements of the present disclosure or the various versions, embodiment(s) or aspects thereof, the articles “a,” “an,” “the” and “said” are intended to mean that there are one or more of the elements. The terms “comprising”, “including” and “having” are intended to be inclusive and mean that there may be additional elements other than the listed elements.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art.

1. A chimeric fusion protein comprising:
a DNA modifying domain fused to a catalytically-inactive Cas (dCas) domain; and
a peptide linker.

2. The chimeric fusion protein of claim 1:
wherein the catalytically-inactive Cas (dCas) domain is a dCas9 domain; and
wherein the dCas9 lacks endonuclease activity.

3. The chimeric fusion protein of claim 1, wherein the DNA modifying domain is selected from the group consisting of an endonuclease, a DNA methyltransferase, a DNA glycosidase, a DNA polymerase, a DNA ligase, a DNA topoisomerase, a DNA kinase, an oxidoreductase, and a histone deacetylase.

4. The chimeric fusion protein of claim 3, wherein the endonuclease is selected from the group consisting of: a type IIS restriction enzyme.

5. The chimeric fusion protein of claim 3, wherein the endonuclease is selected from the group consisting of: FokI, AlwI, BsmFI, BspCNI, BtsCI, HgaI, eco571R, mbollR, and bcgIB.

6. The chimeric fusion protein of claim 3, wherein the DNA methyltransferase is selected from the group consisting of: an N-6 adenine-specific DNA methylase and an N-4 cytosine-specific DNA methylase.

7. The chimeric fusion protein of claim 1, wherein the catalytically inactive Cas (dCas) domain is fused to the C-terminus of the DNA modifying domain via the peptide linker.

8. The chimeric fusion protein of claim 1, wherein the peptide linker comprises between one and one-hundred amino acid residues.

9. The chimeric fusion protein of claim 8, wherein the peptide linker comprises between four and forty amino acid residues.

10. The chimeric fusion protein of claim 1, wherein the peptide linker is selected from the group consisting of: SEQ ID NO: 4, SEQ ID NO: 5, SEQ ID NO: 25, SEQ ID NO: 26, SEQ ID NO: 27, SEQ ID NO: 28, SEQ ID NO: 29, and combinations thereof.

11. The chimeric fusion protein of claim 1, further comprising a nuclear localization signal sequence.

12. An isolated nucleic acid comprising a nucleotide sequence encoding the chimeric fusion protein of claim 1.

13. The isolated nucleic acid of claim 12, further comprising a nucleotide sequence encoding a linker.

14. The isolated nucleic acid of claim 12, further comprising a nucleotide sequence encoding a nuclear localization signal sequence.

15. A vector comprising the nucleic acid of claim 12.

16. The vector of claim 15, further comprising a promoter operably linked to the isolated nucleic acid, wherein the promoter is selected from the group consisting of an inducible promoter and a constitutive promoter.

17. A cell comprising the isolated nucleic acid of claim 16.

18. An organism comprising the isolated nucleic acid of claim 16.

19. A chimeric fusion protein comprising a dCas9 domain fused to a FokI domain, wherein the FokI is relatively at an N-terminus of the dCas9 domain.

20. The chimeric fusion protein of claim 19, further comprising at least one peptide linker.

21. The chimeric fusion protein of claim 20, wherein the peptide linker comprises between one and one-hundred amino acid residues.

22. The chimeric fusion protein of claim 21, wherein the peptide linker comprises between four and forty amino acid residues.

23. The chimeric fusion protein of claim 20, wherein the peptide linker is selected from the group consisting of: SEQ ID NO: 4, SEQ ID NO: 5, SEQ ID NO: 25, SEQ ID NO: 26, SEQ ID NO: 27, SEQ ID NO: 28, SEQ ID NO: 29, and combinations thereof.

24. The chimeric fusion protein of claim 19, further comprising at least one nuclear localization signal sequence.

25. An isolated nucleic acid comprising a nucleotide sequence encoding the chimeric fusion protein of claim 19.

26. The isolated nucleic acid of claim 25, further comprising a nucleotide sequence encoding a peptide linker.

27. The isolated nucleic acid of claim 26, further comprising a nucleotide sequence encoding a nuclear localization signal sequence.

28. A vector comprising the nucleic acid of claim 26.

29. The vector of claim 28, further comprising a promoter operably linked to the isolated nucleic acid, wherein the promoter is selected from the group consisting of an inducible promoter and a constitutive promoter.

30. A cell comprising the isolated nucleic acid of claim 25.

31. An organism comprising the isolated nucleic acid of claim 25.

32. A method of genome editing in a cell, the method comprising:
introducing at least two chimeric fusion protein monomers into a cell, wherein each of the at least two chimeric fusion protein monomers comprises a DNA modifying domain fused to a cleavage-inactive Cas (dCas) domain, and a peptide linker;
introducing a first guide RNA (sgRNA) and a second guide RNA (sgRNA) into the cell,
wherein the first sgRNA and the second sgRNA each comprise an at least 12-20 nucleotide sequence complementary to two adjacent target DNA nucleotide sequences;
wherein two protospacer adjacent motifs (PAM) associated with the two sgRNAs are located outside of the associated sgRNA target site;
wherein the first sgRNA forms a first complex with one chimeric fusion protein monomer and wherein the second sgRNA forms a second complex with one chimeric fusion protein monomer to direct the at least two chimeric fusion protein monomers to the adjacent target DNA nucleotide sequences; and
wherein the DNA modifying domains of the two chimeric fusion protein monomers form a DNA modifying domain dimer; and
inducing a DNA modification in the target DNA using the two chimeric fusion protein monomers.

33. The method of claim 32, wherein the modification to the target DNA is selected from the group consisting of: a double-strand break in the target DNA and a single-strand break in the target DNA.

34. The method of claim 32, further comprising introducing a genetic modification in the target DNA.

35. The method of claim 32, wherein the genetic modification is selected from the group consisting of a DNA deletion, a gene disruption, a DNA insertion, a DNA inversion, a point mutation, a DNA replacement, a knock-in, and a knock-down.

36. The method of claim 32, wherein the cell is selected from the group consisting of a eukaryotic cell and a prokaryotic cell.

37. The method of claim 32 wherein the peptide linker comprises between one and one-hundred amino acid residues.

38. The method of claim 32, wherein the peptide linker comprises between four and forty amino acid residues.

39. The method of claim 32, wherein the peptide linker is selected from the group consisting of: SEQ ID NO: 4, SEQ ID NO: 5, SEQ ID NO: 25, SEQ ID NO: 26, SEQ ID NO: 27, SEQ ID NO: 28, SEQ ID NO: 29, and combinations thereof.

40. The method of claim 32 wherein a spacer length between the first and second sgRNA target sites is from about 1 nucleotide to about 50 nucleotides.

41. The method of claim 40 wherein the spacer length is from 13 nucleotides to 23 nucleotides.

42. The method of claim 40 wherein the spacer length is 30 nucleotides.

43. The method of claim 32 wherein the cell is selected from the group consisting of: a plant cell, an animal cell, an embryo, and a human cell.

44. A method of genome editing in a cell, the method comprising:
introducing at least one FokI-dCas9 fusion protein to the cell;
introducing at least one guide RNA (sgRNA) into the cell, wherein the sgRNA comprises an at least 12-20 nucleotide sequence complementary to a sequence in a target DNA, and guides the FokI-dCas9 fusion protein to the target DNA; and
introducing a different nuclease into the organism, wherein the second nuclease comprises a FokI domain and binds to the adjacent DNA sequence of the sgRNA target site; wherein the second nuclease is a zinc finger nuclease (ZFN), wherein the FokI domain of the FokI-dCas9 chimeric fusion protein and the FokI domain of the ZFN form a FokI dimer and induces a double-strand break in the target DNA.

45. The method of claim 44 wherein the cell is selected from the group consisting of: a plant cell, an animal cell, a embryo, and a human cell.

46. A method of genome editing in a cell, the method comprising:
introducing at least one FokI-dCas9 fusion protein monomer to the cell;
introducing at least one guide RNA (sgRNA) into the cell, wherein the sgRNA comprises an at least 12-20 nucleotide sequence complementary to a sequence in a target DNA, and guides the FokI-dCas9 fusion protein to the target DNA; and
introducing a different nuclease into the organism, wherein the second nuclease comprises a FokI domain and binds to the adjacent DNA sequence of the sgRNA target site; wherein the second nuclease is a Transcription Activator-Like Effector Nuclease (TALEN); wherein the FokI domain of the FokI-dCas9 chimeric fusion protein and the FokI domain of the TALEN form a FokI dimer and induces a double-strand break in the target DNA.

47. The method of claim 46 wherein the cell is selected from the group consisting of: a plant cell, an animal cell, a embryo, and a human cell.


SEQUENCE LISTING (40 sequences)

SEQ ID NO: 1 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 1655 aa, Artificial Sequence
  Synthetic
MDYKDHDGDYKDHDIDYKDDDDKMAPKKKRKVGIHGVPMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVRTGGGSSGTGQGGSAASRGGSLAQDVASTGGGSSGGGPRAGSQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 3 - RNA, 82 nt, Artificial Sequence
  Synthetic
guuuuagagcuagaaauagcaaguuaaaauaaggcuaguccguuaucaacuugaaaaaguggcaccgagucggugcuuuuuu

SEQ ID NO: 4 - protein, 42 aa, Artificial Sequence
  Synthetic
RTGGGSSGTGQGGSAASRGGSLAQDVASTGGGSSGGGPRAGS

SEQ ID NO: 5 - protein, 22 aa, Artificial Sequence
  Synthetic
RTGGGSSGTGGGSSGGGPRAGS

SEQ ID NO: 6 - protein, 7 aa, Simian virus 40
PKKKRKV

SEQ ID NO: 7 - protein, 5 aa, Saccharomyces cerevisiae
KIPIK

SEQ ID NO: 8 - protein, 23 aa, Artificial Sequence
  Synthetic
MDYKDHDGDYKDHDIDYKDDDDK

SEQ ID NO: 9 - protein, 196 aa, Artificial Sequence
  Synthetic
QLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 10 - RNA, 102 nt, Artificial Sequence
  Synthetic
acagaggcuguugguacuagguuuuagagcuagaaauagcaaguuaaaauaaggcuaguccguuaucaacuugaaaaaguggcaccgagucggugcuuuuuu

SEQ ID NO: 11 - RNA, 101 nt, Artificial Sequence
  Synthetic
aaucugcuaguauauccguguuuuagagcuagaaauagcaaguuaaaauaaggcuaguccguuaucaacuugaaaaaguggcaccgagucggugcuuuuuu

SEQ ID NO: 12 - RNA, 102 nt, Artificial Sequence
  Synthetic
cgcccaucuucuagaaagacguuuuagagcuagaaauagcaaguuaaaauaaggcuaguccguuaucaacuugaaaaaguggcaccgagucggugcuuuuuu

SEQ ID NO: 13 - RNA, 102 nt, Artificial Sequence
  Synthetic
uuaaaggcuaaccuggugugguuuuagagcuagaaauagcaaguuaaaauaaggcuaguccguuaucaacuugaaaaaguggcaccgagucggugcuuuuuu

SEQ ID NO: 14 - protein, 5 aa, Homo sapiens
RKRRR

SEQ ID NO: 15 - protein, 5 aa, Mus musculus
KRKRK

SEQ ID NO: 16 - protein, 4 aa, Mus musculus
RRKR

SEQ ID NO: 17 - protein, 5 aa, Mus musculus
RRRRR

SEQ ID NO: 18 - protein, 1607 aa, Artificial Sequence
  Synthetic
MAPKKKRKVGGKPIPNPLLGLDSTHLRGSQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGVPADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 19 - DNA, 4824 bp, Artificial Sequence
  Synthetic
atggcccccaagaagaagaggaaggtcggcggcaagcctatcccaaatccactcctgggtctggacagcactcatctgcggggatcccagctggtgaagagcgagctggaggagaagaagtccgagctgcggcacaagctgaagtacgtgccccacgagtacatcgagctgatcgagatcgccaggaaccccacccaggaccgcatcctggagatgaaggtgatggagttcttcatgaaggtgtacggctacaggggagagcacctgggcggaagcagaaagcctgacggcgccatctatacagtgggcagccccatcgattacggcgtgatcgtggacacaaaggcctacagcggcggctacaatctgcctatcggccaggccgacgagatgcagagatacgtggaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccacatcaccaactgcaatggcgccgtgctgagcgtggaggagctgctgatcggcggcgagatgatcaaagccggcaccctgacactggaggaggtgcggcgcaagttcaacaacggcgagatcaacttcggggtacccgctgacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtga

SEQ ID NO: 20 - protein, 1611 aa, Artificial Sequence
  Synthetic
MAPKKKRKVGGKPIPNPLLGLDSTHLRGSQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAGGAGVPADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 21 - protein, 1608 aa, Artificial Sequence
  Synthetic
MAPKKKRKVGGKPIPNPLLGLDSTHLRGSQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGGVPADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 22 - protein, 1621 aa, Artificial Sequence
  Synthetic
MAPKKKRKVGGKPIPNPLLGLDSTHLRGSQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAGPRGSGNGSSHGAGVPADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 23 - protein, 1643 aa, Artificial Sequence
  Synthetic
MAPKKKRKVGGKPIPNPLLGLDSTHLRGSQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAGPRGSGNQGGSAASTGRGGSLAQRSATGSGSSHGAGVPADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 24 - protein, 196 aa, Planomicrobium okeanokoites
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 25 - protein, 4 aa, Artificial Sequence
  Synthetic
GVPA

SEQ ID NO: 26 - protein, 5 aa, Artificial Sequence
  Synthetic
GGVPA

SEQ ID NO: 27 - protein, 8 aa, Artificial Sequence
  Synthetic
AGGAGVPA

SEQ ID NO: 28 - protein, 18 aa, Artificial Sequence
  Synthetic
AGPRGSGNGSSHGAGVPA

SEQ ID NO: 29 - protein, 40 aa, Artificial Sequence
  Synthetic
AGPRGSGNQGGSAASTGRGGSLAQRSATGSGSSHGAGVPA

SEQ ID NO: 30 - protein, 5 aa, Artificial Sequence
  Synthetic
HLRGS

SEQ ID NO: 31 - protein, 1379 aa, Artificial Sequence
  Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 32 - RNA, 102 nt, Artificial Sequence
  Synthetic
agucuucugggcaggcuuaaguuuuagagcuagaaauagcaaguuaaaauaaggcuaguccguuaucaacuugaaaaaguggcaccgagucggugcuuuuuu

SEQ ID NO: 33 - RNA, 102 nt, Artificial Sequence
  Synthetic
gacuggaguugcagaucacgguuuuagagcuagaaauagcaaguuaaaauaaggcuaguccguuaucaacuugaaaaaguggcaccgagucggugcuuuuuu

SEQ ID NO: 34 - RNA, 102 nt, Artificial Sequence
  Synthetic
gacaucgauguccuccccauguuuuagagcuagaaauagcaaguuaaaauaaggcuaguccguuaucaacuugaaaaaguggcaccgagucggugcuuuuuu

SEQ ID NO: 35 - RNA, 102 nt, Artificial Sequence
  Synthetic
gggcaaccacaaacccacgaguuuuagagcuagaaauagcaaguuaaaauaaggcuaguccguuaucaacuugaaaaaguggcaccgagucggugcuuuuuu

SEQ ID NO: 36 - RNA, 102 nt, Artificial Sequence
  Synthetic
cuccccauuggccugcuucgguuuuagagcuagaaauagcaaguuaaaauaaggcuaguccguuaucaacuugaaaaaguggcaccgagucggugcuuuuuu

SEQ ID NO: 37 - DNA, 886 bp, Mus musculus
ctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagtttta

SEQ ID NO: 38 - DNA, 537 bp, Homo sapiens
cagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactagggtgggcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctccct

SEQ ID NO: 39 - protein, 353 aa, Artificial Sequence
  Synthetic
MDYKDHDGDYKDHDIDYKDDDDKMAPKKKRKVGIHGVPAAMAERPFQCRICMRNFSDRSARTRHIRTHTGEKPFACDICGRKFAQSGHLSRHTKIHTGSQKPFQCRICMRNFSRSDDLSKHIRTHTGEKPFACDICGRKFARNDHRKNHTKIHLRGSQLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 40 - DNA, 144 bp, Artificial Sequence
  Synthetic
ggcctgggagaatcccttccccctcttccctcgtgatctgcaactccagtctttctagaataatacgactcactatagggatccgatgggcgggagtcttctgggcaggcttaaaggctaacctggtgtgtgggcgttgtcctg